SEQUENCE LISTING (6 sequences)

SEQ ID NO: 1 - DNA, 6692 bp, Artificial Sequence
  plasmid BB2224-1
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgccctttctagaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagactgagggagacctaaacacttgaggatcttactgggtgaattgagctgcttagctatggatcccacagttctacccatcaataagtgcttttgtggtagtcttgtggcttccatatctggggagcttcatttgcctttatagtattaaccttctgtgtttaatctctccctcagtcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacactcgag

SEQ ID NO: 2 - DNA, 9217 bp, Artificial Sequence
  plasmid BB2224-2
ggccgcgtgtttcaccgttctcatcttcttgtggctcttggagagcaacaagtgaaactttactcatagtttcttcatgaaatgtgtttaatctctccctcagttcttcaccacatggacagttttctttgtcatcttcttctaaatttttaaaatttatgatagcattcatctgttctcttggagggcaacgaaactctcttgtttttcttgcagcaactgatgatggaagatcagactgcttaatatcaacatatcttctcaactgatcctgctgaagatcagcaatatagtcataactaaaattaataatggcttcaacccgatgccgcaattgtatatcgcataaatgctgtaagagatagcacagcggtgatgactgatgagtgtttaaggaccaatggagagaatgtttgagttgtgaagcggagaacctgaggcgtggttatttatagggaagagaggaaggtgaatgagggacacgtcacagaagtagggtgctgagcttgagacattcttcagtatgcatggctatggaagccttgggtgctacacctcatgaagttcatggtgtgaggtggcttcggcatctcaattaagtgacaaagagaaaggtgtttcagtgtttctattgcaaatggcagaaactcgtgatgacgaggggaccatgcatggtttcatttcttttcttcctggattctttctttccttttatatatgcaggttcataatttaaaaattagactcgctttcaatttcttaatttctcattttcctcttatattactgtactaatgttaaccacgtacacttattttttttttagtttaattttgatagattgtgttgatttaaacatattaatattttcaaccaaataaaaatcattttagtagatacggctttttaaataattattaaaaatattaactatttatcctaaatggcacattttaattaaaaaaaatccggtgttgtaagtgttttattaatttgttttggcattattaaagcaactttttttttatttgttggcattttgagtacgtacttaggctagccgcggccgccagtgtgatggatatcagctgggccggccactagaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcggtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatgacctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatccatggctgtagagtcaacaatcacaagataaatcagaatcaaaatccatcacgtaatttcaagaagcaatttcgtgaaaaaaaatcctaatctatggccacagaatcaacaaataaggtagttgctaaaggacctgagcaatatctcttgaaataccatacagaatcaacgaataaggcttttatcaacggatctgaacaatttcttgaaaaacgactgaagtatcaacaaataaggctgttgtttaacggatctgaagtagttcatgaaaaaaaccttaatctacgactacagaatcaacaattaaggcttgttgtaaacggatctgaaaatctttatgaaaaaaactctaattagacctaatttacggttataaaatcatcaattaaggacgttcttgcattaataaaagctgaatcttctgattatgaaaatttataatcgatgaaacgatagaaaacacaatcaaatcaacatcatgctgatgtagaacaaacaataaagaagaagaaaaaaaaacggaagaaatctacgaaccttgaaaggagaggtagggaactgcgcaaagagaatgaactaatttgattgcaattgaagtgcgattagggtcgaggggtgtgggggcattttatagcatatgcggtggaaccgttctccaatcatattgtgacgcgtgcccatgtatggtgtgacttggtgtttatgacacagatgatatttcggggagtccttcgtagttgacggtttcgtaatcttgacggagtcagagatgtaccttcaccgcttcacgcttggctttatccgttaccacggcgttattaaattgggcccgcacccgttgcaagttaggaccacagtgtccattacctgaacggtcggctgctaccagtattttacattattaaaatctatggaaaagctgtcacgctagatttttttttttttttttatggataagaaaagactaataggaaagctttcttactccactaaagtctttaataattattttttcaattttcaaataaaatcataatcatcccactaaattctttctcatttgtttttcactttgtaagatattcttccgtagatttttataagtaattctttaagatacatagcataataaattgtatacttttagttacagtctatataaaattagtcatttaaaaaatatagccttaatgatttttttaaaatgaagttatttttttctaatttttctttcttatttattatcgaatgattttttctactaatcgtttagggaagagttttatttttctaatgacgagatgtgttaaatgatgcaacttgaaggcgacatattttttaccaataaaataaaactaaaagatagcttgttgacaaaaacaataattgatgttaaagaaaacgtaagggcaatgggtatcatctaattgttggacaagttgtcttctaaggctaggtaagcttttgttttttattagctgaaatatttatttaattgttctataaataaattttttaaaatgatttttaatgtttttaaatattatttaaaataatttttttaaaattttaacattttagtttttaattattttttatcttacttaaattaattataatttattattttctaccattttatactttccaattaattttatcgaagcattaataccatctggtattttgtcactgcttcgtatgattgtttattaaattttagcttctttcacattggagatgctgggcggtgtgagttacagcatggtgtgaagttaaggtggtttgattatggaacccgaaggtgttggtgcaaggtggataaaattgcttgccaggagatacataaaatgatttacaaaatgaaataaagaataaataaatagtcaagttcaaatttaaatcaaggattagaattatttttccgtacatgatgtaaccgcaatcacccgggtcgagatatctagacccagctttcttgtacaaagtggttcgataaacactagacccatcagctgaggtacaccggtagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgccctaggcgctatcaactttgtatagaaaagttggtgtttcaccgttctcatcttcttgtggctcttggagagcaacaagtgaaactttactcatagtttcttcatgaaatgtgtttaatctctccctcagttcttcaccacatggacagttttctttgtcatcttcttctaaatttttaaaatttatgatagcattcatctgttctcttggagggcaacgaaactctcttgtttttcttgcagcaactgatgatggaagatcagactgcttaatatcaacatatcttctcaactgatcctgctgaagatcagcaatatagtcataactaaaattaataatggcttcaacccgatgccgcaattgtatatcgcataaatgctgtaagagatagcacacaactttattatacaaagttgatagatatcggtccgagatccatcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtccatggtggagctcgaccgatatctatcaactttgtataataaagttgtgtgctatctcttacagcatttatgcgatatacaattgcggcatcgggttgaagccattattaattttagttatgactatattgctgatcttcagcaggatcagttgagaagatatgttgatattaagcagtctgatcttccatcatcagttgctgcaagaaaaacaagagagtttcgttgccctccaagagaacagatgaatgctatcataaattttaaaaatttagaagaagatgacaaagaaaactgtccatgtggtgaagaactgagggagagattaaacacatttcatgaagaaactatgagtaaagtttcacttgttgctctccaagagccacaagaagatgagaacggtgaaacaccaacttttctatacaaagttgatagcgttaacccgggtaactgtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatcgatgggcccgtaccggccggcctctgcctgcgttctgctgtggaagttcctattccgaagttcctattctccagaaagtataggaacttcacatgctgcctcgtgcaagtcacgatctcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtacccgggtaccgagctcactagacgcggtgaaattacctaattaacaccggtgtttatcaaacaagtttgtacaaaaaagcaggctggcgccggaaccaattcagtcgagtgc

SEQ ID NO: 3 - DNA, 9792 bp, Artificial Sequence
  plasmid BB2224-3
ctagaagcttttgatccatgcccttcatttgccgcttattaattaatttggtaacagtccgtactaatcagttacttatccttcccccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagccctcacatttgttgtttctaacccaacctcaaactcgtattctcttccgccacctcatttttgtttatttcaacacccgtcaaactgcatgccaccccgtggccaaatgtccatgcatgttaacaagacctatgactataaatagctgcaatctcggcccaggttttcatcatcaagaaccagttcaatatcctagtacaccgtattaaagaatttaagatatactgcggccgcttctagctagctagggtttgggtagtgagtgtaataaagttgcaaagtttttggttaggttacgttttgaccttattattatagttcaaagggaaacattaattaaaggggattatgaagactgagggagacctaaacacttgaggatcttactgggtgaattgagctgcttagctatggatcccacagttctacccatcaataagtgcttttgtggtagtcttgtggcttccatatctggggagcttcatttgcctttatagtattaaccttctgtgtttaatctctccctcagtcacccttctcttcttttctctcataataatttaaatttgttatagactctaaactttaaatgttttttttgaagtttttccgtttttctcttttgccatgatcccgttcttgctgtggagtaaccttgtccgaggtatgtgcatgattagatccatacttaatttgtgtgcatcacgaaggtgaggttgaaatgaactttgcttttttgaccttttaggaaagttcttttgttgcagtaatcaattttaattagttttaattgacactattacttttattgtcatctttgttagttttattgttgaattgagtgcatatttcctaggaaattctcttacctaacattttttatacagatctatgctcttggctcttgcccttactcttggccttgtgttggttatttgtctacatatttattgactggtcgatgagacatgtcacaattcttgggcttatttgttggtctaataaaaggagtgcttattgaaagatcaagacggagattcggttttatataaataaactaaagatgacatattagtgtgttgatgtctcttcaggataatttttgtttgaaataatatggtaatgtcttgtctaaatttgtgtacataattcttactgattttttggattgttggatttttataaacaaatctgcggccgcaagtatgaactaaaatgcatgtaggtgtaagagctcatggagagcatggaatattgtatccgaccatgtaacagtataataactgagctccatctcacttcttctatgaataaacaaaggatgttatgatatattaacactctatctatgcaccttattgttctatgataaatttcctcttattattataaatcatctgaatcgtgacggcttatggaatgcttcaaatagtacaaaaacaaatgtgtactataagactttctaaacaattctaaccttagcattgtgaacgagacataagtgttaagaagacataacaattataatggaagaagtttgtctccatttatatattatatattacccacttatgtattatattaggatgttaaggagacataacaattataaagagagaagtttgtatccatttatatattatatactacccatttatatattatacttatccacttatttaatgtctttataaggtttgatccatgatatttctaatattttagttgatatgtatatgaaagggtactatttgaactctcttactctgtataaaggttggatcatccttaaagtgggtctatttaattttattgcttcttacagataaaaaaaaaattatgagttggtttgataaaatattgaaggatttaaaataataataaataacatataatatatgtatataaatttattataatataacatttatctataaaaaagtaaatattgtcataaatctatacaatcgtttagccttgctggacgaatctcaattatttaaacgagagtaaacatatttgactttttggttatttaacaaattattatttaacactatatgaaatttttttttttatcagcaaagaataaaattaaattaagaaggacaatggtgtcccaatccttatacaaccaacttccacaagaaagtcaagtcagagacaacaaaaaaacaagcaaaggaaattttttaatttgagttgtcttgtttgctgcataatttatgcagtaaaacactacacataacccttttagcagtagagcaatggttgaccgtgtgcttagcttcttttattttatttttttatcagcaaagaataaataaaataaaatgagacacttcagggatgtttcaacactcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtacccgggtaccgagctcactagacgcggtgaaattacctaattaacaccggtgtttatcaaacaagtttgtacaaaaaagcaggctggcgccggaaccaattcagtcgagtgcggccgcgtgtttcaccgttctcatcttcttgtggctcttggagagcaacaagtgaaactttactcatagtttcttcatgaaatgtgtttaatctctccctcagttcttcaccacatggacagttttctttgtcatcttcttctaaatttttaaaatttatgatagcattcatctgttctcttggagggcaacgaaactctcttgtttttcttgcagcaactgatgatggaagatcagactgcttaatatcaacatatcttctcaactgatcctgctgaagatcagcaatatagtcataactaaaattaataatggcttcaacccgatgccgcaattgtatatcgcataaatgctgtaagagatagcacagcggtgatgactgatgagtgtttaaggaccaatggagagaatgtttgagttgtgaagcggagaacctgaggcgtggttatttatagggaagagaggaaggtgaatgagggacacgtcacagaagtagggtgctgagcttgagacattcttcagtatgcatggctatggaagccttgggtgctacacctcatgaagttcatggtgtgaggtggcttcggcatctcaattaagtgacaaagagaaaggtgtttcagtgtttctattgcaaatggcagaaactcgtgatgacgaggggaccatgcatggtttcatttcttttcttcctggattctttctttccttttatatatgcaggttcataatttaaaaattagactcgctttcaatttcttaatttctcattttcctcttatattactgtactaatgttaaccacgtacacttattttttttttagtttaattttgatagattgtgttgatttaaacatattaatattttcaaccaaataaaaatcattttagtagatacggctttttaaataattattaaaaatattaactatttatcctaaatggcacattttaattaaaaaaaatccggtgttgtaagtgttttattaatttgttttggcattattaaagcaactttttttttatttgttggcattttgagtacgtacttaggctagccgcggccgccagtgtgatggatatcagctgggccggccactagaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcggtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatgacctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatccatggctgtagagtcaacaatcacaagataaatcagaatcaaaatccatcacgtaatttcaagaagcaatttcgtgaaaaaaaatcctaatctatggccacagaatcaacaaataaggtagttgctaaaggacctgagcaatatctcttgaaataccatacagaatcaacgaataaggcttttatcaacggatctgaacaatttcttgaaaaacgactgaagtatcaacaaataaggctgttgtttaacggatctgaagtagttcatgaaaaaaaccttaatctacgactacagaatcaacaattaaggcttgttgtaaacggatctgaaaatctttatgaaaaaaactctaattagacctaatttacggttataaaatcatcaattaaggacgttcttgcattaataaaagctgaatcttctgattatgaaaatttataatcgatgaaacgatagaaaacacaatcaaatcaacatcatgctgatgtagaacaaacaataaagaagaagaaaaaaaaacggaagaaatctacgaaccttgaaaggagaggtagggaactgcgcaaagagaatgaactaatttgattgcaattgaagtgcgattagggtcgaggggtgtgggggcattttatagcatatgcggtggaaccgttctccaatcatattgtgacgcgtgcccatgtatggtgtgacttggtgtttatgacacagatgatatttcggggagtccttcgtagttgacggtttcgtaatcttgacggagtcagagatgtaccttcaccgcttcacgcttggctttatccgttaccacggcgttattaaattgggcccgcacccgttgcaagttaggaccacagtgtccattacctgaacggtcggctgctaccagtattttacattattaaaatctatggaaaagctgtcacgctagatttttttttttttttttatggataagaaaagactaataggaaagctttcttactccactaaagtctttaataattattttttcaattttcaaataaaatcataatcatcccactaaattctttctcatttgtttttcactttgtaagatattcttccgtagatttttataagtaattctttaagatacatagcataataaattgtatacttttagttacagtctatataaaattagtcatttaaaaaatatagccttaatgatttttttaaaatgaagttatttttttctaatttttctttcttatttattatcgaatgattttttctactaatcgtttagggaagagttttatttttctaatgacgagatgtgttaaatgatgcaacttgaaggcgacatattttttaccaataaaataaaactaaaagatagcttgttgacaaaaacaataattgatgttaaagaaaacgtaagggcaatgggtatcatctaattgttggacaagttgtcttctaaggctaggtaagcttttgttttttattagctgaaatatttatttaattgttctataaataaattttttaaaatgatttttaatgtttttaaatattatttaaaataatttttttaaaattttaacattttagtttttaattattttttatcttacttaaattaattataatttattattttctaccattttatactttccaattaattttatcgaagcattaataccatctggtattttgtcactgcttcgtatgattgtttattaaattttagcttctttcacattggagatgctgggcggtgtgagttacagcatggtgtgaagttaaggtggtttgattatggaacccgaaggtgttggtgcaaggtggataaaattgcttgccaggagatacataaaatgatttacaaaatgaaataaagaataaataaatagtcaagttcaaatttaaatcaaggattagaattatttttccgtacatgatgtaaccgcaatcacccgggtcgagatat

SEQ ID NO: 4 - DNA, 12 bp, Artificial Sequence
  RYN71 microRNA
ggaaccccccag

SEQ ID NO: 5 - DNA, 363 bp, Nezara viridula
tgtgctatctcttacagcatttatgcgatatacaattgcggcatcgggttgaagccattattaattttagttatgactatattgctgatcttcagcaggatcagttgagaagatatgttgatattaagcagtctgatcttccatcatcagttgctgcaagaaaaacaagagagtttcgttgccctccaagagaacagatgaatgctatcataaattttaaaaatttagaagaagatgacaaagaaaactgtccatgtggtgaagaactgagggagagattaaacacatttcatgaagaaactatgagtaaagtttcacttgttgctctccaagagccacaagaagatgagaacggtgaaacac

SEQ ID NO: 6 - DNA, 15780 bp, Nezara viridula
caacttcctaacgacgaggtagttcttggatatgtaatacgggagagccatccacttctttcactggtctgctaagtagagaggatggccgacagcgaaggaggatccgagcaggacgatgtttcgttcctgaggacggaggatatggtgtgcctatcatgcacagcaactggagagagagtttgcttagcagctgagggctttggtaaccgtcactgttttctagaaaatattgctgataagaatataccaccagatctttcaacatgtgtatttgttattgaacaagctctatcagtaagagcacttcaggagttagttacagcagctggatctgaagagggaaagggaactggatctggtcacaggactcttctttatggaaatgctatactactccggcaccaaaacagtgacatgtatctggcttgtttatctaccagttcatcaaatgacaagctctcatttgatgttggtttacaagaacattcccaaggggaagcttgttggtggaccgtacaccctgcttctaaacagagatcagaaggtgaaaaagtgagagttggtgatgatttaattcttgtgtctgtagccactgaaagatatttgcatactgctaaagaaaacgatcaatctattgtaaatgcatctttccatgtaactcattggtctgttcagccttatggaactggtatcagcaaaatgaagtatgttggttatgtgttcggaggagatgtgttaagatttttccatggtggggatgaatgccttaccattccatcaacttggagtgaaacccctggacaaaatgtggtagtttatgaaggagggagtgttttgagtcaagctcgttcactttggagattggaactggctaggacaaaatggtctggtggtttcattaattggtatcatccaatgaggatacgacatctcaccactggtagatacttaggagttaatgaaaataatgaattacacctcgttgttagggaggaagccacaacagcattatctacattcattttaagacaagaaaaagatgaccaaaaagtagtaatggaagataaggatttagaagtaataggagctccaataataaaatatggtgacagtactgttttagtccaacattcagaaagtggtttatggttaacttataagtcattcgaaactaagaaaaaaggtgtgggtaaagtagaagaaaaacaagctgtacttcatgaggagggaaaaatggatgatggattagactttagtagaagtcaagaagaagaatcaaggactgctagagtaataaggaaatgttcgtcacttttcactcaatttattaggggtctagaaactctgcaaatgaatcgaagacattctctgttttgcgctagtgtaaatttaaatgaaatggtcatgtgtttagaagatttaattaattactttgcccagcctgaggaagatatggaacatgaggaaaaacaaaaccggttaagagctttgagaaacagacaagatttgttccaagaagaaggaattttaaatcttatcttagaagccattgataaaattaatgttataacatcccaaggtttcttagtcagtttagctggagatgagtctggacagagctgggatataatctcaggatatttgtatcaactgctagctgccatcataaaaggaaatcatactaattgtgctcagtttgctaacacaaatagattaaactggttatttagcagactaggttctcaagcttcaagtgagggcacaggtatgttggatgtacttcattgcgtcttaattgattctccagaagctttgaatatgatgagagatgaacatataaaagtaatcatttcactgctagaaaaacatgggcgagatccaagagttttagatgtactttgttcactttgtgttggtaatggtgtagcagtccgtagctcacaaaacaacatctgtgatttccttctgccaggaaaaaacttgcttctacaaacgcaacttgtggatcatgttgccagtgtcaggccaaatatttttgtgggtcgagtcgaaggttctgctgtttatcaaaaatggtattttgaagtgactttagatcatatggagcaaaccacccatatgacaccgcatctaagaattggctgggctaacacttctggttatgttccctttcctggcggtggtgaaaaatggggcggtaatggagttggtgatgatctctactcttttggttttgatggagctgcattatggacaggtggaagaaaaactgtagtccttcctcatgctatggaaccttacataagaaagggagatgttattggttgtgctttcgatctgactgttccaattattacatttacttttaatggaacattaatccgaggatcatttagggattttaatcttcaaggaatgttctttccagttataagctgttcctcaaaacttagttgtcgttttttactgggaggtgatcatggaagattaaaatatgcacctcctgaagaattttctcctctcgttgaaagtttgcttcctcaacaagtgctttctattgatccatgtttttattttggcaacctgaataaatgtgtattggctggtccttatcctgttgaagatgattgtgcttttgttccagttccagttgacacatctatggtaaatttacccgttcatgttgatacaatacgcgatcgtttagctgaaaacatccatgaaatgtgggctatgaataaaattgaagcaggatggatttatggagatgtaagagatgatataagaagaatacatccatgtcttgtgcaatttgaaaaactacctcctgcagaaaagcgatatgacactcaacttgctgtacaaactttaaaaaccatcattgcactgggctaccatataacaatggaaaaaccaccatctagaataaagaacattcgtttgccgaatgaaccatttttacaatctaatggttacaagccagctcctcttgatctcagtgccataacactaatacctaaaatggaggaacttgttgaccaactcgctgaaaatactcacaacttgtgggcaaaagaaagaatccaacaaggctggacctatggtcttaatgaggatcctgatttgtcccgaagtcctcacctcgtcccttacagtaaagttgatgatttaattaaaaaagccaacagggataccgcaagtgaaactgtcaggactcttcttgtttatggttataatttagaccctcctacaggtgaacaaactgaagctctcttagcagaagcaagccgtttgaagcagatgcagtttagaacctatcgggctgaaaagacatatgcagtaaccagtggcaaatggtattttgaatttgaaattcttactgctgggccaatgagagtaggttgggccattgctgattataatccaggttcccagatcggaagtgatgaagcatcctgggcatatgatggttataatgaggaaaaggtttattctggggttgctgaaacgtttggaagacaatggcaagttggagacgttgtaggagtttttcttgatctattggatcatactattagtttctctctaaatggtgaactgcttatggatgcacttgggggagaaacatcttttgcagatgttcagggagaaggatttgttccagcatttacacttggagtaggacaaaaagcaaaattagtgtttgggcaagatgttaactcacttaagttctttactacctgtggtttgcaagaaggttatgaacctttctgtgtaaacatgaacagggcagttaccttttggtacaccaaagatcatcctatatttgaaaatactgatgattatattgatactaaaattgatgcaacgcgtattcctgctggttctgacacaccaccatgtcttaaaattagtcataatacttttgagacaatggagaaagccaattgggaatttcttagactttctttacctgttcaatgtttaccatcattcataaatgaacaagaaaaagtacgtaggtggcaagaaataaggataagacaacacagacttcttgtggaagctgaccaaaccactcctgctcacattgaacagattatgaagtctggttttagtatgagtgatattaagggtcttcaaagaagttatacagaagatggaatggaaggagaagaaggattggcaccaagctcatcaccacttacaaggactaagtcaaaagtgactccagctcgtccacctaggaaaggctccttaccacgaaatggagatgttattaatatgaacgggacattagaaccaggtggaggaaaaatgaaccgttctaatagtgagcttgatttccaacgtttcaatggtgaaatgcccgatggcgataacaagaaaaagcgtgggagatctccatttaggttcttttcaagaaaaaagggggagcgtgatactagtggagaaaatgcaaaaaatgtacatatgtctgagcctatgggtaatttccttgagcctccaaggactccaatgcagcaaagaggtggaagtgctctgcgttcttctcctcaacctaaagtacaggagttaactaagccaccatccccattagttgaaagaagtggacccaaagcaatgtctgtgcctgttggaactggcatcgaaactattggaaatgaaatatttgatgtagagtgtttgaaattgattaatgaatacttctacggtgtcaggatatttccaggtcaagacccaactcatgtatatgtcggttgggttacaactcaattccatctacgtagtaaagactttaatcagaatcgagtgctaaagagcactgtagtagtatgtgatgaattcaatcgtgtaatagacagtattcagcggcagagttgttttatggtaagagctgatgaattatacaatcaagtaactcaggatgcctctggtaaaggtgcttcacaaggaatgtttattggatgtttcctggatactgctactggttatgtgacgttcacatgtgaaggaaaagaaactaaccacaagtataagatggaacctgatacaaaattatttccagctatatttgttgaagctacaagcaaagaaattctacaaattgagcttggtcgtacatcaactacactgcctttatcagcagctgttctccaaaattcagaaagacatgtcattcctcagtttccaccaagacttaaagttcagtgtctaaaaccacatcagtgggcacgtgttcctaatatttcattgcatgtccacgctctgaaattatcagatataagaggttggagtatgctttgtgaagatccagtttcaatgttagcattacatatacctgaagaagatagatgtattgatattttagaacttattgaaatggacaaactactttcattccatgctcatacattgacactttatgcagcactatgttaccaatccaattatcgtgcaggacatgttctctgcaaacatgtagaccaaaagcaacttcagtatgctattaggtctgaattcatatctggatctttacgcttgggattttatgacctcttgattgctttacacattgaatcacatgcaacaacaatggaagtttgtaaaaatgaattcataataccccttggtctagacttgaaagatttatatgaagatccagatatgaagcacagcttacgatctttaaaaactgtctctattttacctcaaatgagtatgacagacattacggaaaatattgaaagcatcaatacattatatagtccttattttcctcttgatgcagttaaggattatggaatgactgcattagaagaggctgtaagcatgaatcaacttcacaatagagaccctgtaggtggttcaaatgaaaacttgtttctacccttgttgaaactggtagatagattattgcttgttgggatactacgagatgaagatgttacaaagctactaattatgtttgatcctgaaacttgggattcaaattttgaaaaggatggcaaagatgaacatcgtaagggtttacttcaaatgaaaatggcagagggggcaaaactacagatgtgctatctcttacagcatttatgcgatatacaattgcggcatcgggttgaagccattattaattttagttatgactatattgctgatcttcagcaggatcagttgagaagatatgttgatattaagcagtctgatcttccatcatcagttgctgcaagaaaaacaagagagtttcgttgccctccaagagaacagatgaatgctatcataaattttaaaaatttagaagaagatgacaaagaaaactgtccatgtggtgaagaactgagggagagattaaacacatttcatgaagaaactatgagtaaagtttcacttgttgctctccaagagccacaagaagatgagaacggtgaaacaccagaaaagccgggtgttttcaaaaaattatacaattttattaatgctgttaaagaattggaagaacctcctaaaatagaagaagaacctgttaagaaaactcctgaagaaatatttagaaaagtattaattagtacaattgttagatgggctgaagaatcccagattgaaacaccaaaattagtcagagaaatgttcagtctattggtaaggcagtacgacactgtaggtgaattaatcagatctcttggaaacacttatgtgataaatgacaaaacgaaagaagatgtagctcagatgtgggtagggttgagccagatcagagctctcctacctgttcaaatgtctcaagatgaagaaggtcttatgcgaatgaggctatggaaattagttaacaatcacacattctttcaacatcctgatttgattagagttcttcgtgttcatgaaaatgttatggctgttatgatcaataccttgggtagaagatcacaagcacaatctgatgcttctcaagctggtcaagaaggtgaacctgcagctaaggagaaagatacgtcccatgaaatggtggtagcatgttgtcgtttcctgtgttatttttgcagaacttcacgtcaaaatcagaaagcaatgtttgaccatttaacatttttattagaaaacagtaatattttactttcaagaccttcacttagaggaagtacccctcttgatgttgcctattcctctctcatggaaaataccgaactggcattagctcttagagaacattatttagagaagatagctgtttacttgtctcgctgtggattacaatctaattcagaattggtagaaaagggttaccctgatttgggttgggatccagttgagggagaaagatatttagactttttacgcttctgtgtttgggttaacggtgaaagtgttgaagaaaatgcaaatctggttatacggctccttatacgtcgaccagaatgtttgggtcctgcacttcgtggagaaggtgaaggattactgagagcaattatagatgctaataagatgtctgaaagaatttcagatcgcagaaaaatgatggaggaacctgaaaattctgcccatcatcagtttgaacatccacttcctgagtctgatgaagatgaggactatattgatacaggagcagcaatactggcattctattgtactctggtcgatcttttaggtcgctgtgctccagatgctagtgtgattgctcagggaaagaatgagtctcttagagctagagctattttgagatctttagtacctcttgaagatttatttggtgtcttgagtttaaagtttacacttaccaatccagctattggagaagaaaggccaaaaagtgatataccatctggtctaataccatctcataagcaaagtattgttttatttttagagagagtatatggtattgaacagcaagatctcttcttcagattactcgaggaagcatttttacctgatttaagagcagcaactatgctagatagaactgatggttctgaatcagaaatggcattagctatgaatcgctatattggaaattctattctccctttgttgataaagcattaccagttttatagtggtgcagataactatgcaagtcttttagatgctacacttcatacagtgtatcgcctatcaaaaaatcgaatgctaactaaaggtcagcgagaggcagtatcagattttttggttgctctcacaagtcaattacagccaagcatgttactcaaacttcttcgaaagttaaccgttgatgtatcaaagctttctgagtataccacagttgctttaaggttgcttactttacactatgagcgttgtgcaaaatattatggaactactggtggacaagctggtggatctagtgatgaagaaaaaaggctcactatgttactcttcagtaatatttttgattctttatcaaaaatggattatgatcctgaattatttggaaaagcgcttccctgcttgagtgctataggatgtgcacttccacccgattattcactgtccaagaattatgatgaagaatggtatagttcaaagggttcagaaccgactgatgggccttataatccactgcccatcaatacttctatggtttctctaaataatgatttaaacacaattgttcaaaaattttctgaacattatcatgatgcatgggctagtcgaaaaatggaaaatggttgggtatatggtgagcagtggtctgacagctctaaaactcatcctcgtttaaaaccttatacattgcttaatgattatgaaaaagagagatacaaagaaccggttagagagtcattgaaagctctgttagctataggatggaatgtagagcatactgaagttgatattccttctaataacagaggatcatcagtcagaagatcttctaaagcaaatacatctgatggttcaacaccatttaattatcatcccaacccaattgatatgactaatttaacattgagtagagaaatgcaaaatatggcagagaggttagctgaaaactcacatgatatttgggcaaaaaagaagaaagaagaacttgtttcatgtggtggtggtatacacccacagcttgttccatatgatcttttaacagacaaagagaagaggaaagatagagaaagatctcaagaatttttgaaatatttacaatatcaaggatacaaactccacaggcctactcgaggaagtgctgatgagcaacaggccgctgcagctgctgccacaggagagtccagatttgcttacagtctactcgagaaacttatacaatatactgataaagcttctattaatatgaaactactaaagccttctggtacattcagtagacgctccagttttaaaacttgttcaagagacataaaattcttttccaaagtggtattgctattggttgagaagtatttcagcactcacagaaattacttcattgctgttgccactgcttctaataatgtaggagcagcctctttaaaagaaaaagaaatggttgccagtttgttctgtaagctggcaaatttaattcgaacaaagctggctgcttttggtgcagatgttcgaattactgtccgttgtctacaagtgctagtgaaagctatagatgccaagtcattggtaaagaattgtcctgaatttataaggacttcaatgctgacatttttcaataatacagctgatgacttaggccaaactattcagtgtttgcaagagggtcgttacagtcaccttagaggcactcatcttaaaacatctacttctttattttatataaatgatgttgtactacctgttctcacttctatgtttgatcatttggctgtgtgtgattatggtagcgacttgttacttgatgaaattcaagtggcctcatatagaatgttgggtagtttatataatttaggaattgatccaactttaactcatgacagaaaatatttaaaaacagaaattgaaaggcataggcctgccattggtgcttgtcttggtgcattttcatcaacatttccagtcgcttatcttgaaccccatttaaataaacataatcagttttcattagttaatagaattgctgaacattctcttgaagcacaggatattctagctagaatggaaaacaccatgcctacattggatgcgatcctttctgaagttgatcagttcattgaatccgaaaagagtcatacttcagcaccacatgttattgatgtgattttgcctctgctttgtgcttatttgccaagttggtggagtcaaggtcctgataatgtcagtctcacagcagggaattatgtaacaatggttactagtgatcatatgaatcaactcctaaaaaatgtactaaaattaatcaaaaataatattggaaatgaaaatgctccctggatgacgagaatagcagcttacacccagcagatcatcataaactcttctgaagaactgttgaaagatccattccttccattaacacaagttgttaagaagaggatagacaatatgtttcaccgtgaagaatctcttcgaggatttctaaaatcttcaactgaagatacctctcaagttgaagcagaaattcaggagggctggcatcttattgttagagatatatattctttttatccactactaattaaatatgttgatttacaaagaaatcactggttacgtaataatattccggaagctgaatacttgtatactcatgttgctgatatatttaatatttggtctaaatcacagtactttctaaaagaagaacagaatttcatatctgccaacgaaatagacaatatggctctaattatgcccactgcaactaggagatctgcagttgttttggatggaacagctcctgctggaggtggaaagaagaaaaagaagcatcgtgataagaaaagagataagaataaagaaatccaagcaagcttaatggtagcttgcttaaaacgtttattaccagttggtcttaacctattcgctggaagagaacaagagttagttcagcattgtaaagacagatatttgaagaaaatgccagaatatgaaatagtggattttgccaaaatccaattaactcttcctgacaagatagatcctggagatgagatgtcttggcagcattatttgtactcaaaactgggaaataaaaaagatatcagctctgaaaaaccacagcaaatcgatgaggtagttgataggattgtggctatggcaaaagttctttttgggcttcatatgattgatcatccacaactacagagcaagacacaatacagatctgttgtatccacacagagaaagcgtgctgtcatagcttgtttccggcaactatcactacatgccttaccaagcatgcaaataaacctccacctcaccaatctggatggaaaagagttctttcagcagcgagaaaacgggctgctattgcttgtcttagaactcaacctttgtatacccttccaaggcatcgagtaattaacatatttgctcgcgcttattgtgagctgtggctgcaagaagagaatgttggtcaagaaatcatgattgaagatcttacacaaacttttgaagatgctgaattgaaaaaaagagattctgaagaagatgaaagcaaacctgatccacttacccaattagttacaacattttgtcggggtgcaatgactgaaaggagtggagctttgcaagaagacccactttatatgtcctatgcagaaattactgcaaaatcatgtggagaagaagaagaagaaggtggagatgaggaagaaggtggagacgaagaaggaggggcatctatccataagacaatggcaaaattagtggaacaagaaatggaaaaacagaaactcttattccatcaagctcggctagccaacagaggtgttgcagaaatggtattgttacatatttcagcttgtaaaggtgttcccagtgaaatggttatgaaaactctccagctgggtatttctgttttacgtggtggtaatcttgatattcaaatgggtatgctaaatcatttgaaagaaaaaaaggatgttggattttttacttctatagctggcttgatgaactcctgcagtgtgttggatttagatgcatttgaaagaaacacaaaagctgaaggcttaggagttggttcagaaggtgctgctggtgaaaagaacatgcatgatgctgaattcacctgtactcttttcagatttattcaacttacctgtgaagggcataacttagaatggcagaattatcttagaacccaagctggaaatacaacaacagttaatgttgttatttgtactgttgattaccttttgagattacaggaatcaattatggacttctattggcactattcgagtaaagaattaattgatcctgctggaaaagccaactttttcaaagcaattggtgtggctagtcaagtatttaatacactctctgaagtaattcaagggccttgcccacaaaatcaacaagctctggctcattcaagattgtgggatgctgttggaggatttttgtttcttttctctcatatgcaagataagctatcaaaacattctagtcaagtagacttactgaaagaacttttgaatttacagaaagatatgataacaatgatgctatcaatgttggaaggtaatgttgtgaatggtactattggaaaacagatggtagacacattagttgaatctgcctcaaatgtggaattgattttgaagtacttcgacatgtttttgaaattgaaagatttgacatcctctgctagcttcttggaacttgatccaaaccatgaaggctgggtaacacctaaagattttaaagaaaaaatggaacagcagaaaagttatactccagaagaaatagacttcatgttacagtgctgtgaaaccaatcatgacggtaaaattgactatgttggcttcacggatagattccatgagccggccaaggaaattggttttaacctagctgttcttctcacaaatttatctgagcatatgccaaatgaaccgagacttgctcgctttttagaaacagctggtagtgttcttaactactttgaacctttcctgggacgaattgaaatattaggtagtagtaaacgaatcgagcgtgtatatttcgagattaaagaatcaaatattgaacagtgggaaaaacctcaaatcaaggaatctaaacgagcatttttctattcaattgtcactgaaggaggtgacaaagaaaaattggaagcttttgttaatttttgtgaagatgccatatttgagatgacacatgccagtgggcttatggcaactgatgatggtacaggctctggaggaggaaaacaaagagcatcctcttattcttatatggaagatgaagatgaagaaaggaatccaatcagacgtggttggcaagcaactaaagatggaatttactttatgttctcaatgttatctcctagcaatattaaacataaaattattgaaatgcaacaaatgtcaattattgaactaatgattggttttataaaactatttttctacatgttttattactcaggatattctgtatcagttgtactgaagtatattggtggtattatattttcattgatgaggggaccacaaattgaagagccagttgtagaagttaaagaggaagaaaaatctggacctctgaggataatgcctgctttgccaccacctgaagatagctctctgcttccatctgatgggtcaagagacatgaaaaaagaagacagtcagcctccatcaaaagtcatagaaggggctattcccatagaagaaggaggtgagaggagctcagaggaacatgcgggagaccatgtaaaaccagaaaatgaagagcaacctccaacaccaacacttgctgatatattgggtggagaagcagcaagaaaagaagcagcacaaagagcagaagtcgctgctgaacaagaagcagttatggctgcttttgaggcagaatctaaaatagaaaaagtttcagagccttctgctgtctctcaaattgattttaacaagtatactcaccgggctgtcagtttccttgctcgtaatttctataatcttaaatatgtagcattggttttggctttctgcattaactttattttattgttctacaaggtaacaacattgggtgaagatgatgatgctgctagcggagaagggagtgttgaacaactaatggaagaattaacaggcgaaggtgatgatgtgagtggcggaggaagtagtggtggagaaagtggtgaagaggatccaattgaaatggttcatgtggatgaggatttcttttatatggcacatgttatgcgattggctgcaatcctacattctcttgtttctttagctatgttgattgcatattatcatttgaaggtccctctagctatattcaagagagaaaaagaaatagctcgtcgacttgagtttgatggtttgtacattgctgagcaaccagaagatgatgatattaaatcacattgggataaactggttatctgtgcaaaatcatttcctgttaattactgggataaatttgtgaagaaaaaggttcgacagaaatacagtgaaacttatgactttgattcaataagtaatcttttgggaatggaaaaaacatctttcagtgcccaagatactgaagaaggatcgggacttattcattacattttgaactttgactggaggtatcagctttggaaagcaggagtcacaatcacagataatgcatttttgtacagtttattatacttcatcttttcaattttgggaaacttcaataactttttctttgctgcccatttacttgatgttgcagttggttttaaaacattgaggactattttgcaatcagtcacacacaatggaaaacagcttgtattgactgtaatgctgctaaccatcatagtatacatctatactgtcattgctttcaacttcttccgaaaattttatgtccaagaagaggatgaggaagtggataaaaaatgccacgatatgttaacttgttttgtattccacctttacaaaggagttagagctggtggtggtattggtgatgagattgaacctcctgatggtgatgattatgaagtttacaggataatgtttgatattacgtttttcttttttgttattgtcatcttgctagccatcattcaaggtttgatcattgatgcatttggtgaattgagagatcagttagaaagtgtaaaagaagacatggaatctaactgcttcatttgtgggataggaaaagattattttgataaagttccccatggttttgacactcatgttcaacaagaacataacttggctaattacatgttctttcttatgcatctgattaacaagccagatactgaatacacaggtcaagaaacctatgtctggaacatgtatcagcaacgttgttgggatttcttcccagttggtgactgttttcgtaaacagtatgaagatgaactgggaggtggtggtggttaattcatttgggtgggtggtggctaaatttatattattaaaacaaaattaatgctgggaactatcaaacatccttcaattttattaaaatttcagctaaattcaacaatatatcttatgatattgtatttgtctaatgaaggaatagaactatcgtgttatgaatcagtgaagttttcacttgtttagcataatttatgctaagtttactattgcaaaatactttctttatatccgaaaatgttgtaaaataaatgtaaatggtgtggccttaaatataatg

(19) United States
(12) Patent Application Publication
(10) Pub. No.: US 2016/0024518 A1
(43) Pub. Date: Jan. 28, 2016

(54) PRODUCTION OF SMALL INTERFERING RNAS IN PLANTA

(71) Applicants: Brian MCGONIGLE, Johnston, IA (US); E.I. DUPONT DE NEMOURS & COMPANY, Wilmington, DE (US)

(72) Inventor: BRIAN MCGONIGLE, WILMINGTON, DE (US)

(21) Appl. No.: 14/774,612

(22) PCT Filed: Mar. 13, 2014
(86) PCT No.: PCT/US2014/026448

§ 371 Date: Sep. 10, 2015

Related U.S. Application Data
(60) Provisional application No. 61/779,840, filed on Mar. 13, 2013.

(51) Int. Cl. C12N 15/82; A01H 5/00

(52) U.S. Cl. CPC C12N 15/8279; A01H 5/00

(57) ABSTRACT

Disclosed herein are methods for reducing the expression of target nucleotide sequences using microRNAs. Also disclosed herein are recombinant DNA constructs comprising nucleotide sequences encoding such microRNAs. Plants or plant part thereof comprising these recombinant DNA constructs and microRNAs are also provided.

### REFERENCE TO SEQUENCE LISTING SUBMITTED ELECTRONICALLY

The official copy of the sequence listing is submitted electronically as an ASCII formatted sequence listing with a file named “BB2224WOPCT_Sequence_Listing.TXT” and is filed concurrently with the specification. The sequence listing contained in this ASCII formatted document is part of the specification and is herein incorporated by reference in its entirety.

### TECHNICAL FIELD

This disclosure relates generally to the field of plant molecular biology. More specifically, it relates to constructs and methods to reduce the level of expression of a target sequence in a target organism.

### BACKGROUND

A wide variety of eukaryotic organisms, including plants, animals, and fungi, have evolved several RNA-silencing pathways to protect their cells and genomes against invading nucleic acids, such as viruses or transposons, and to regulate gene expression during development or in response to external stimuli (for review, see Baulcombe (2005) Trends Biochem. Sci. 30:290-93; Meins et al. (2005) Annu. Rev. Cell Dev. Biol. 21:297-318). In plants, RNA-silencing pathways have been shown to control a variety of developmental processes including flowering time, leaf morphology, organ polarity, floral morphology, and root development (reviewed by Mallory and Vaucheret (2006) Nat. Genet. 38:S31-36). All RNA-silencing systems involve the processing of double-stranded RNA (dsRNA) into small RNAs of 21 to 25 nucleotides (nt) by an RNaselll-like enzyme known as Dicer or Dicer-like in plants (Bernstein et al. (2001) Nature 409:363-66; Xie et al. (2004) PLOS Biol. 2 E104:0642-52; Xie et al. (2005) Proc. Natl. Acad. Sci. USA 102:12984-89; Dunoyer et al. (2005) Nat. Genet. 37:1356-60). These small RNAs are incorporated into silencing effector complexes containing an Argonaute protein (for review, see Meister and Tuschl (2004) Nature 431:343-49).

Artificial microRNAs (amiRNAs) have been described in Arabidopsis targeting viral mRNA sequences (Niu et al. (2006) Nat. Biotechnol. 24:1420-1428) or endogenous genes (Schwab et al. (2006) Plant Cell 18:1121-1133). The amiRNA construct can be expressed under different promoters in order to change the spatial pattern of silencing (Schwab et al. (2006) Plant Cell 18:1121-1133). Artificial miRNAs replace the microRNA and its complementary star sequence in a miRNA precursor backbone and substitute sequences that target an mRNA to be silenced. Silencing by endogenous miRNAs can be found in a variety of spatial, temporal, and developmental expression patterns (Parizotto et al. (2007) Genes Dev. 18:2237-2242; Alvarez et al. (2006) Plant Cell 18:1134-51).

Traditionally, the primary method for impacting pest populations is the application of broad-spectrum chemical pesticides. However, consumers and government regulators alike are becoming increasingly concerned with the environmental hazards associated with the production and use of synthetic chemical pesticides. Because of such concerns, regulators have banned or limited the use of some of the more hazardous pesticides. Thus, there is substantial interest in developing alternative pesticides.

### SUMMARY

Applicant has solved the problem through development of recombinant DNA constructs comprising 1) a precursor miRNAs that, when fully processed, yield 22 nucleotide mature miRNAs that is capable of triggering the production of secondary siRNAs in planta targeted to at least one exogenous gene, and 2) a polynucleotide sequence that includes at least one exogenous target site that can be cleaved by the 22 nucleotide miRNA. These siRNAs can, when ingested by a target organism, result in gene silencing of the targeted gene(s).

One aspect is for a method for reducing expression of at least one target sequence, said method comprising: (a) expressing in a plant a recombinant DNA construct comprising: (i) a first polynucleotide sequence comprising a plant-specific promoter operably linked to a nucleotide sequence encoding a pre-miRNA, wherein said pre-miRNA comprises a 22 nucleotide mature miRNA; and (ii) a second polynucleotide sequence comprising at least one target sequence that can be cleaved by the mature mi-RNA processed by the pre-miRNA of (i), wherein said plant processes said pre-miRNA into mature miRNA and; (b) eliciting production of of secondary siRNAs in planta by the mature miRNA; wherein exposing a target organism to said plant comprising the secondary siRNAs of step (b), reduces expression of at least one target sequence in said target organism.

Another aspect is for a recombinant DNA construct comprising: (a) a first polynucleotide sequence comprising a plant-specific promoter operably linked to a nucleotide sequence encoding a pre-miRNA, wherein said pre-miRNA comprises a 22 nucleotide mature miRNA; and (b) a second polynucleotide sequence comprising at least one exogenous target sequence that can be cleaved by the mature mi-RNA processed by the pre-miRNA of (a); wherein the mature miRNA elicits the production of secondary siRNAs.

A further aspect is for a plant or a plant part thereof comprising: (a) a first recombinant DNA construct comprising a first plant-specific promoter operably linked to a polynucleotide encoding a first portion of a pre-miRNA, said first portion of a pre-miRNA comprising a first polynucleotide segment of 22 nucleotides; and (b) a second recombinant DNA construct comprising a second plant-specific promoter operably linked to a polynucleotide encoding a second portion of a pre-miRNA, said second portion of a pre-miRNA comprising a second polynucleotide segment complementary to said first polynucleotide segment; wherein said first polynucleotide segment has sufficient sequence complementary to at least one target sequence whose level of RNA is to be reduced but does not have sufficient sequence complementary to any RNAs of a plant expressing the recombinant DNA constructs; and further wherein said first polynucleotide sequence, when processed into a mature miRNA, elicits the production of secondary siRNAs.

An additional aspect is for a plant or plant part thereof comprising: (a) a first polynucleotide comprising a plant-specific promoter operably linked to a nucleotide sequence encoding a pre-miRNA, wherein said pre-miRNA comprises a 22 nucleotide mature miRNA; and (b) a second polynucleotide sequence comprising at least one exogenous target sequence that can be cleaved by the mature mi-RNA processed from the pre-miRNA of (a); wherein said mature miRNA, elicits the production of secondary siRNAs.

### BRIEF DESCRIPTION OF THE FIGURES

FIG. 1 shows the plasmid BB2224-1.

FIG. 2 shows the plasmid BB2224-2.

FIG. 3 shows the plasmid BB2224-3.

### BRIEF DESCRIPTION OF THE BIOLOGICAL SEQUENCES

The following sequences comply with 37 C.F.R. §§1.821-1.825 (“Requirements for Patent Applications Containing Nucleotide Sequences and/or Amino Acid Sequence Disclosures-the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5(a-bis), and Section 208 and Annex C of the Administrative Instructions. The symbols and format used for nucleotide and amino acid sequence data comply with the rules set forth in 37 C.F.R. §1.822.

SEQ ID NO:1 is the nucleotide sequence of plasmid BB2224-1.

SEQ ID NO:2 is the nucleotide sequence of plasmid BB2224-2.

SEQ ID NO:3 is the nucleotide sequence of plasmid BB2224-3.

SEQ ID NO:4 is a nucleotide sequence corresponding to the 22 base pair microRNA sequence targeting the ryodine 7 fragment (RYN7a).

SEQ ID NO:5 is the nucleotide sequence of the RYN7a fragment of the Southern green stinkbug Nezara viridula (Linnaeus).

SEQ ID NO:6 is the nucleotide sequence of a ryodine gene of the Southern green stinkbug Nezara viridula (Linnaeus).

### DETAILED DESCRIPTION

Compositions and methods are provided comprising a recombinant DNA construct comprising a first polynucleotide sequence comprising a plant-specific promoter operably linked to a nucleotide sequence encoding a pre-miRNA, wherein said pre-miRNA comprises a 22 nucleotide mature miRNA; and a second polynucleotide sequence comprising at least one exogenous target sequence that can be cleaved by the mature mi-RNA processed by the pre-miRNA; wherein the mature miRNA elicits the production of secondary siRNAs in planta. These siRNAs can, when ingested by the target organism, result in gene silencing of the targeted gene.

In this disclosure, a number of terms and abbreviations are used. The following definitions apply unless specifically stated otherwise. As used herein, the articles “a”, “an”, and “the” preceding an element or component of the disclosure are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

The term “comprising” means the presence of the stated features, integers, steps, or components as referred to in the claims, but does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

“Mature MicroRNA” or “mature miRNA” refers to oligoribonucleic acid, generally of 19, 20, 21, 22, 23, or 24 nucleotides (nt) in length, which regulates expression of a polynucleotide comprising a target sequence. Mature microRNAs are non-protein-coding RNAs and have been identified in both animals and plants (Lagos-Quintana et al., Science 294:853-858 (2001), Lagos-Quintana et al. (2002) Curr. Biol. 12:735-739; Lau et al. (2001) Science 294:858-862; Lee and Ambros (2001) Science 294:862-864; Llave et al. (2002) Plant Cell 14:1605-1619; Mourelatos et al. (2002) Genes Dev. 16:720-728; Park et al. (2002) Curr. Biol. 12:1484-1495; Reinhart et al. (2002) Genes Dev. 16:1616-1626). Mature miRNAs are derived, in plants, via dicer-like 1 processing of larger precursor polynucleotides. As discussed in further detail elsewhere herein, an miRNA can be an “artificial miRNA” or “amiRNA” which comprises an miRNA sequence that is synthetically designed to silence a target sequence.

Plant miRNAs regulate endogenous gene expression by recruiting silencing factors to complementary binding sites in target transcripts. MicroRNAs are initially transcribed as long polyadenylated RNAs (“pri-miRNA”) and are processed to form a shorter sequence that has the capacity to form a stable hairpin (“pre-miRNA”) and, when further processed by the siRNA machinery, release a miRNA. In plants, both processing steps are carried out by Dicer-like nucleases. miRNAs function by base-pairing to complementary RNA target sequences and trigger RNA cleavage of the target sequence by an RNA-induced silencing complex (“RISC”).

A small RNA interaction with a target sequence can trigger the production of secondary small interfering RNAs (“siRNAs”) from the regions surrounding their primary target sites (Sijen et al. (2001) Cell 107(4):465-76). Secondary amplification of the siRNA population, and thus amplification of the level of gene silencing, occurs via an RNA-dependent RNA polymerase (“RDR”)-dependent and Dicer-dependent pathway that uses the primary target RNA as a template to generate secondary siRNAs. Newly synthesized double stranded RNA (“dsRNA”) is subsequently cleaved into siRNAs that are able to guide the degradation of additional secondary target RNAs in a sequence-independent manner. Transitive silencing via this process of generating secondary siRNAs has been observed only in plants and Caenorhabditis elegans and only with siRNA and double stranded RNA constructs (Sijen et al., supra; Vaistij et al. (2002) Plant Cell 14:857-867).

As used herein, an “miRNA precursor backbone” is a polynucleotide that provides the backbone structure necessary to form a hairpin RNA structure which allows for the processing and ultimate formation of the miRNA. Thus, the miRNA precursor backbones are used as templates for expressing artificial miRNAs and their corresponding star sequence. Within the context of an miRNA expression construct, the miRNA precursor backbone comprises a DNA sequence having the heterologous miRNA and star sequences. When expressed as an RNA, the structure of the miRNA precursor backbone is such as to allow for the formation of a hairpin RNA structure that can be processed into an miRNA. In some embodiments, the miRNA precursor backbone comprises a genomic miRNA precursor sequence, wherein said sequence comprises a native precursor in which a heterologous miRNA and star sequence are inserted.

As used herein, a “star sequence” is the sequence within an miRNA precursor backbone that is complementary to the mature miRNA and forms a duplex with the mature miRNA to form the stem structure of a hairpin RNA. In some embodiments, the star sequence can comprise less than 100% complementarity to the mature miRNA sequence. Alternatively, the star sequence can comprise at least 99%, 98%, 97%, 96%, 95%, 90%, 85%, 80% or lower sequence complementarity to the mature miRNA sequence as long as the star sequence has sufficient complementarity to the mature miRNA sequence to form a double stranded structure. In still further embodiments, the star sequence comprises a sequence having 1, 2, 3, 4, 5 or more mismatches with the mature miRNA sequence and still has sufficient complementarity to form a double stranded structure with the mature miRNA sequence resulting in production of mature miRNA and suppression of the target sequence.

A “target sequence” refers to the sequence that the mature miRNA is designed to reduce and thus the expression of its RNA is to be modulated, e.g., reduced. The region of a target sequence of a gene of interest which is used to design the mature miRNA may be a portion of an open reading frame, 5′ or 3′ untranslated region, exon(s), intron(s), flanking region, etc. General categories of genes of interest include, for example, those genes involved in information, such as transcription factors, those involved in communication, such as kinases, and those involved in housekeeping, such as heat shock proteins.

An “exogenous target sequence” is a target sequence which is foreign to the plant hosting a recombinant DNA construct of the present disclosure.

By “sufficient sequence complementarity” to the target sequence is meant that the complementarity is sufficient to allow the mature miRNA to bind to a target sequence and reduce the level of expression of the target sequence. In specific embodiments, a miRNA having sufficient complementarity to the target sequence can share 100% sequence complementarity to the target sequence or it can share less than 100% sequence complementarity (i.e., at least 99%, 98%, 97%, 96%, 95%, 90%, 85%, 80%, 75%, 70% or less sequence complementarity) to the target sequence. In other embodiments, the miRNA can have 1, 2, 3, 4, 5 or up to 6 alterations or mismatches with the target sequence, so long as the mature miRNA has sufficient complementarity to the target sequence to reduce the level of expression of the target sequence. Endogenous miRNAs with multiple mismatches with the target sequence have been reported. For example, see Schawb et al. (2005) Dev. Cell 8:517-27, and Cuperus et al. (2010) Nat. Struct. Mol. Biol. 17:997-1003, herein incorporated by reference in their entirety.

When designing a mature miRNA sequence and star sequence for the miRNA expression constructs disclosed herein, various design choices can be made. See, for example, Schwab R et al. (2005) Dev. Cell 8: 517-27. In non-limiting embodiments, the mature miRNA sequences disclosed herein can have a “U” at the 5′-end, a “C” or “G” at the 19^{th} nucleotide position, and an “A” or “U” at the 10th nucleotide position. In other embodiments, the miRNA design is such that the mature miRNA have a high free delta-G as calculated using the ZipFold algorithm (Markham, N. R. & Zuker, M. (2005) Nucleic Acids Res. 33:W577-W581). Optionally, a one base pair change can be added within the 5′ portion of the mature miRNA so that the sequence differs from the target sequence by one nucleotide.

As used herein, by “controlling a pest” or “controls a pest” is intended any effect on a pest that results in limiting the damage that the pest causes. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant.

Assays that measure the control of a pest are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. See, for example, Thomma et al. (1998) Plant Biol. 95:15107-15111, herein incorporated by reference.

As used herein, “reducing,” “suppression,” “silencing,” and “inhibition” are used interchangeably to denote the down-regulation of the level of expression of a product of a target sequence relative to its normal expression level in a wild-type organism. By “reducing the level of RNA” is intended a reduction in expression by any statistically significant amount including, for example, a reduction of at least 10%, 15%, 20%, 25%, 30%, 35%, 40%, 45%, 50%, 55%, 60%, 65%, 70%, 75%, 80%, 85%, 90%, 95%, or 100% relative to the wild type expression level. The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

The terms “polynucleotide,” “polynucleotide sequence,” “nucleic acid sequence,” and “nucleic acid fragment” are used interchangeably herein. These terms encompass nucleotide sequences and the like. A polynucleotide may be a polymer of RNA or DNA that is single-or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. The use of the term “polynucleotide” is not intended to limit the present disclosure to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the disclosure also encompass all forms of sequences including, but not limited to, single-stranded forms, double-stranded forms, hairpins, stem-and-loop structures, and the like.

The compositions provided herein can comprise an isolated or substantially purified polynucleotide. An “isolated” or “purified” polynucleotide is substantially or essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived.

Further provided are recombinant polynucleotides comprising the miRNA expression constructs and various components thereof. The terms “recombinant polynucleotide” and “recombinant DNA construct” are used interchangeably herein. A recombinant construct comprises an artificial or heterologous combination of nucleic acid sequences, e.g., regulatory and coding sequences that are not found together in nature. For example, an miRNA expression construct can comprise an miRNA precursor backbone having heterologous polynucleotides comprising the miRNA sequence and the star sequence, and thus the miRNA sequence and star sequence are not native to the miRNA precursor backbone. In other embodiments, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the constructs disclosed herein. The skilled artisan will also recognize that different independent transformation events will result in different levels and patterns of expression (Jones et al. (1985) EMBO J. 4:2411-2418; De Almeida et al. (1989) Mol. Gen. Genet. 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

In specific embodiments, one or more of the miRNA expression constructs described herein can be provided in an expression cassette for expression in a plant or other organism or cell type of interest. The cassette can include 5′ and 3′ regulatory sequences operably linked to a polynucleotide provided herein. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of a recombinant polynucleotide to be under the transcriptional regulation of the regulatory regions. The expression cassette may additionally contain selectable marker genes.

In one embodiment, the present disclosure encompasses a method to produce siRNAs in planta. The method uses a recombinant construct comprised of two parts. The first part of the construct is a primary miRNA which produces a 22 nucleotide mature miRNA. It is known to those skilled in the art that 22 nucleotide miRNAs cause the production of secondary siRNAs (Published US patent application 2012/0297508). The second part of the construct is a polynucleotide sequence that includes a non-endogenous target site that can be cleaved by the 22 nucleotide miRNA produced from the first part. The presence of both the 22 nucleotide mature miRNA as well as an appropriate exogenous target sequence within the plant causes the production of siRNAs within the plant. These siRNAs can, when ingested by the target organism, result in gene silencing of the targeted gene.

In another embodiment, the present disclosure encompasses a method to produce siRNAs in planta. The method uses two recombinant constructs wherein a first recombinant construct comprises a primary miRNA which produces a 22 nucleotide mature miRNA and a secondary recombinant construct comprising a polynucleotide sequence that includes an exogenous target sequence that can be cleaved by the 22 nucleotide miRNA produced from the recombinant construct. The first and second recombinant constructs can be driven by the same promoter or different promoters. The method will only function in the temporal/spatial time in which the two promoter activities are coincident. The exogenous target site could be, e.g., homologous to a gene in, e.g., some insect, fungal, or weed pest.

In another embodiment, the present disclosure encompasses a method to produce siRNAs in planta wherein the method uses two recombinant constructs as described above and wherein the second recombinant construct comprises multiple exogenous target sequences with homology to more than one gene, or other polynucleotide target.

As used herein, “heterologous” with respect to a sequence is intended to mean a sequence that originates from a foreign species, or, if from the same species, is substantially modified from its native form in composition and/or genomic locus by deliberate human intervention. For example, with respect to a nucleic acid, it can be a nucleic acid that originates from a foreign species, or is synthetically designed, or, if from the same species, is substantially modified from its native form in composition and/or genomic locus by deliberate human intervention. Thus, in the context of a miRNA expression construct, a heterologous miRNA and star sequence are not native to the miRNA precursor backbone.

As used herein, “plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores. The term “plant part” includes differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant part may be in plant or in a plant organ, tissue or cell culture.

A transformed plant or transformed plant cell provided herein is one in which genetic alteration, such as transformation, has been affected as to a gene of interest, or is a plant or plant cell which is descended from a plant or cell so altered and which comprises the alteration. A “transgene” is a gene that has been introduced into the genome by a transformation procedure. Accordingly, a “transgenic plant” is a plant that contains a transgene, whether the transgene was introduced into that particular plant by transformation or by breeding; thus, descendants of an originally-transformed plant are encompassed by the definition. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which does not express the miRNA, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the miRNA; or (e) the subject plant or plant cell itself, under conditions in which the miRNA expression construct is not expressed.

The terms “introducing” and “introduced” are intended to mean providing a nucleic acid (e.g., miRNA expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a miRNA expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

“Stable transformation” is intended to mean that the nucleotide construct introduced into a host (i.e., a plant) integrates into the genome of the plant and is capable of being inherited by the progeny thereof. “Transient transformation” is intended to mean that a polynucleotide is introduced into the host (i.e., a plant) and expressed temporally.

“Variants” refer to substantially similar sequences. For polynucleotides, a variant comprises a deletion and/or addition of one or more nucleotides at one or more internal sites within the polynucleotide and/or a substitution of one or more nucleotides at one or more sites in the polynucleotide. Variants of the miRNA expression constructs, miRNA precursor backbones, miRNAs, and/or star sequences disclosed herein may retain activity of the miRNA expression construct, miRNA precursor backbone, miRNA, and/or star sequence as described in detail elsewhere herein. Variant polynucleotides can include synthetically derived polynucleotides, such as those generated, for example, by using site-directed mutagenesis. Generally, variants of a miRNA expression construct, miRNA precursor backbone, mature miRNA, and/or star sequence (e.g., SEQ ID NO:4) disclosed herein will have at least about 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482-489; the global alignment algorithm of Needleman and Wunsch (1970) J. Mol. Biol. 48:443-453; the search-for-local alignment method of Pearson and Lipman (1988) Proc. Natl. Acad. Sci. USA 85:2444-2448; the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence provided herein. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See, e.g., the Pubmed website. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix. By “equivalent program” is intended any sequence comparison program that, for any two sequences in question, generates an alignment having identical nucleotide or amino acid residue matches and an identical percent sequence identity when compared to the corresponding alignment generated by GAP Version 10.

“Target organism” refers to an organism that contains at least one target sequence for the mature miRNAs disclosed herein. A target organism is exogenous to an organism, typically a plant, that initially processes a precursor miRNA of the present disclosure into a mature miRNA. In some embodiments, the target organism is an animal, plant, or fungi. Target animals can be invertebrates or vertebrates, with arthropods, particularly insects, being target organisms of interest.

Insect targets include, but are not limited to, targets for Lepidoptera, Diptera, Coleoptera, and Hemiptera order organisms. In some embodiments, the insect targets are Pentatomidae plant pests (stink bugs and shield bugs) or Nezara viridula, Acrosternum hilare, Piezodorus guildini, and/or Halymorpha halys plant pests or inducing resistance in a plant to a plant pest, such as Pentatomidae plant pests or N. viridula, Acrosternum hilare, Piezodorus guildini, and/or Halymorpha halys plant pests. As used herein “Pentatomidae plant pest” is used to refer to any member of the Pentatomidae family. Accordingly, the compositions and methods are also useful in protecting plants against any Pentatomidae plant pest including representative genera and species such as, but not limited to, Acrocorisellus (A. serraticollis), Acrosternum (A. adelpha, A. hilare, A. herbidum, A. scutellatum), Agonoscelis (A. nubila), Alcaeorrhynchus (A. grandis, A. phymatophorus), Amaurochrous (A. brevitylus), Apateticus (A. anatarius, A. bracteatus, A. cynicus, A. lineolatus, A. marginiventris), Apoecilus, Arma (A. custos), Arvelius, Bagrada, Banasa (B. calva, B. dimiata, B. grisea, B. induta, B. sordida), Brochymena (B. affinis, B. cariosa, B. haedula, B. hoppingi, B. sulcata), Carbula (C. obtusangula, C. sinica), Chinavia, Chlorochroa (C. belfragii, C. kanei, C. norlandi, C. senilis, C. viridicata), Chlorocoris (C. distinctus, C. flaviviridis, C. hebetatus, C. subrugosus, C. tau), Codophila (C. remota, C. sulcata, C. varius), Coenus (C. delius, C. inermis, C. tarsalis), Cosmopepla (C. bimaculata, C. binotata, C. carnifex, C. decorata, C. intergressus), Dalpada (D. oculata), Dendrocoris (D. arizonesis, D. fruticicola, D. humeralis, D. parapini, D. reticulatus), Dolycoris (D. baccarum (sloe bug)), Dybowskyia (D. reticulata), Edessa, Erthesina (E. fullo), Eurydema (E. dominulus, E. gebleri (shield bug), E. pulchra, E. rugosa), Euschistus (E. biformis, E. integer, E. quadrator, E. servus, E. tristigma), Euthyrhynchus (E. floridanus, E. macronemis), Gonopsis (G. coccinea), Graphosoma (G. lineatum (stink bug), G. rubrolineatum), Halyomorpha (H. halys (brown marmorated stink bug)), Halys (H. sindillus, H. sulcatus), Holcostethus (H. abbreviatus, H. fulvipes, H. limbolarius, H. piceus, H. sphacelatus), Homalogonia (H. obtusa), Hymenarcys (H. aequalis, H. crassa, H. nervosa, H. perpuncata, H. reticulata), Lelia (L. decempunctata), Lineostethus, Loxa (L. flavicollis, L. viridis), Mecidea (M. indicia, M. major, M. minor), Megarrhamphus (M. hastatus), Menecles (M. insertus, M. portacrus), Mormidea (M. cubrosa, M. lugens, M. pama, M. pictiventris, M. ypsilon), Moromorpha (M. tetra), Murgantia (M. angularis, M. tessellata, M. varicolor, M. violascens), Neottiglossa (N. califomica, N. cavifrons, N. coronaciliata, N. sulcifrons, N. undata), Nezara (N. smaragdulus, N. viridula (southern green stink bug)), Oebalus (O grisescens, O insularis, O mexicanus, O pugnax, O typhoeus), Oechalia (O schellenbergii (spined predatory shield bug)), Okeanos (O quelpartensis), Oplomus (O catena, O dichrous, O tripustulatus), Palomena (P. prasina (green shield bug)), Parabrochymena, Pentatoma (P. angulata, P. illuminata, P. japonica, P. kunmingensis, P. metallifera, P. parataibaiensis, P. rufipes, P. semiannulata, P. viridicornuta), Perillus (P. bioculatus, P. confluens, P. strigipes), Picromerus (P. griseus), Piezodorus (P. degeeri, P. guildinii, P. lituratus (gorse shield bug)), Pinthaeus (P. humeralis), Plautia (P. crossota, P. stali (brown-winged green bug)), Podisus (P. maculiventris), Priassus (P. testaceus), Prionosoma, Proxys (P. albopunctulatus, P. punctulatus, P. victor), Rhaphigaster (R. nebulosa), Scotinophara (S. horvathi), Stiretrus (S. anchorago, S. fimbriatus), Thyanta (T. accerra, T. calceata, T. casta, T. perditor, T. pseudocasta), Trichopepla (T. aurora, T. dubia, T. pilipes, T. semivittata, T. vandykei), Tylospilus, and Zicrona.

In some embodiments the insect targets are Chrysomelidae, particularly corn rootworm (Diabrotica virgifera, D. barberi, D. undecimpunctata howardi). In some embodiments, the corn rootworm is a western corn rootworm (D. virgifera virgifera).

MicroRNA expression constructs encoding a 22-nucleotide (22-nt) mature miRNA are provided herein. As used herein, an miRNA expression construct comprises a polynucleotide capable of being transcribed into an RNA sequence which is ultimately processed in the cell to form a mature miRNA. In some embodiments, the miRNA encoded by the miRNA expression construct is an artificial miRNA. Various modifications can be made to the miRNA expression construct to encode a mature miRNA. Such modifications are discussed in detail elsewhere herein.

The miRNA precursor backbones can be from any plant. In some embodiments, the miRNA precursor backbone is from a monocot. In other embodiments, the miRNA precursor backbone is from a dicot. In further embodiments, the backbone is from maize or soybean. MicroRNA precursor backbones have been described previously. For example, US20090155910A1 (WO 2009/079532) discloses the following soybean miRNA precursor backbones: 156c, 159, 166b, 168c, 396b and 398b, and US20090155909A1 (WO 2009/079548) discloses the following maize miRNA precursor backbones: 159c, 164h, 168a, 169r, and 396h. Each of these references is incorporated by reference in their entirety. It is recognized that some modifications can be made to the miRNA precursor backbones provided herein, such that the nucleotide sequences maintain at least 60%, 70%, 75%, 80%, 85%, 90%, 95%, 96%, 97%, 98%, 99% or more sequence identity with the nucleotide sequence of the unmodified miRNA precursor backbone. Such variants of an miRNA precursor backbone retain miRNA precursor backbone activity and thereby continue to allow for the processing and ultimate formation of the miRNA.

When designing an miRNA expression construct to target a sequence of interest, the miRNA sequence of the backbone can be replaced with a heterologous miRNA designed to target any sequence of interest. In such instances, the corresponding star sequence in the miRNA expression construct will be altered such that it base pairs with the designed miRNA sequence in the precursor RNA to form an imperfect stem structure. In such instances, both the star sequence and the miRNA sequence are heterologous to the miRNA precursor backbone.

Thus, in one embodiment, the miRNA precursor backbone can be altered to allow for efficient insertion of new miRNA and star sequences within the miRNA precursor backbone. In such instances, the miRNA segment and the star segment of the miRNA precursor backbone are replaced with the heterologous miRNA and the heterologous star sequences using a PCR technique and cloned into an expression plasmid to create the miRNA expression construct. It is recognized that there could be alterations to the position at which the heterologous miRNA and star sequences are inserted into the backbone. Detailed methods for inserting the miRNA and star sequence into the miRNA precursor backbone are described in, for example, US Patent Applications 20090155909A1 and US20090155910A1, herein incorporated by reference in their entirety.

In one embodiment, the miRNA precursor backbone comprises a first polynucleotide segment encoding a miRNA and a second polynucleotide segment encoding a star sequence, wherein the first and second polynucleotide segments are heterologous to the miRNA precursor backbone.

The order of the miRNA and the star sequence within the miRNA expression construct can be altered. For example, in specific embodiments, the first polynucleotide segment comprising the miRNA segment of the miRNA expression construct is positioned 5′ to the second polynucleotide sequence comprising the star sequence. Alternatively, the second polynucleotide sequence comprising the star sequence can be positioned 5′ to the first polynucleotide sequence comprising the miRNA sequence in the miRNA expression construct.

As discussed above, the miRNA expression constructs are designed such that the mature miRNA produced from the miRNA expression construct is 22-nt in length. Such an expression construct will therefore comprise a first polynucleotide segment comprising the miRNA sequence and a second polynucleotide segment comprising the corresponding star sequence, wherein the star sequence has at least 1-nt less than the polynucleotide encoding the corresponding miRNA. In such instances, having at least 1 less nucleotide in the star sequence will create a mismatch or “bulge” in the miRNA sequence when the star sequence and miRNA sequence hybridize to each other. Such a structure results in a 22-nt miRNA being the most abundant form of miRNA produced. See, Cuperus et al. (2010) Nat. Struct. Mol. Biol. 17(8):997-1004, herein incorporated by reference in its entirety. As used herein, by “most abundant form” is meant the 22-nt miRNA represents the largest population of miRNAs produced from the miRNA expression construct. In other words, while the miRNA expression construct may produce miRNAs that are not 22-nt in length (i.e. 19-nt, 20-nt, 21-nt, etc.) the most abundant miRNA produced from the miRNA expression construct is 22-nt in length. Thus, the 22-nt miRNA represents at least 50%, 60%, 70%, 80%, 90%, 95% or 100% or the total miRNA population produced from the miRNA expression construct.

The 22-nt miRNA produced from the miRNA expression construct is capable of reducing the level of expression of the target sequence and reducing the level of mRNA of the target sequence and at least one additional sequence from the same protein and/or gene family, the members of which would not be reduced by a 21-nt miRNA directed to the same region as the 22-nt miRNA. Methods to assay for reduction in expression of two or more members of a protein and/or gene family include, for example, monitoring for a reduction in mRNA levels from the same protein and/or gene family or monitoring for a change in phenotype. Various ways to assay for a reduction in the expression of two or more members of a protein and/or gene family are discussed elsewhere herein. Thus, as disclosed herein, a single miRNA can silence multiple proteins/genes in a protein and/or gene family or an entire protein and/or gene family.

The expression cassette can include, in the 5′-3′ direction of transcription, a transcriptional and translational initiation region (i.e., a promoter), a recombinant polynucleotide provided herein, and a transcriptional and translational termination region (i.e., termination region) functional in plants. The regulatory regions (i.e., promoters, transcriptional regulatory regions, and translational termination regions) and/or a recombinant polynucleotide provided herein may be native/analogous to the host cell or to each other. Alternatively, the regulatory regions and/or a recombinant polynucleotide provided herein may be heterologous to the host cell or to each other. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, the regulatory regions and/or a recombinant polynucleotide provided herein may be entirely synthetic.

The termination region may be native with the transcriptional initiation region, may be native with the operably linked recombinant polynucleotide of interest, may be native with the plant host, or may be derived from another source (i.e., foreign or heterologous) to the promoter, the recombinant polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

In preparing the miRNA expression cassette, the various DNA fragments may be manipulated so as to provide for the DNA sequences in the proper orientation. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the miRNA expression constructs provided herein. The promoters can be selected based on the desired outcome. It is recognized that different applications can be enhanced by the use of different promoters in the miRNA expression constructs to modulate the timing, location and/or level of expression of the miRNA. Such miRNA expression constructs may also contain, if desired, a promoter regulatory region (e.g., one conferring inducible, constitutive, environmentally-or developmentally-regulated, or cell-or tissue-specific/selective expression), a transcription initiation start site, a ribosome binding site, an RNA processing signal, a transcription termination site, and/or a polyadenylation signal.

In some embodiments, an miRNA expression construct provided herein can be combined with constitutive, tissue-preferred, or other promoters for expression in plants. Examples of constitutive promoters include the cauliflower mosaic virus (CaMV) 35S transcription initiation region, the 1′-or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill. If low level expression is desired, weak promoter(s) may be used. Weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No. 6,177,611, herein incorporated by reference.

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the ERE promoter which is estrogen induced, and the Axig1 promoter which is auxin induced and tapetum specific but also active in callus (WO 2002/006499).

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promoter, Boronat, A. et al. (1986) Plant Sci. 47:95-102; Reina, M. et al. (1990) Nucleic Acids Res. 18(21):6426; and Kloesgen, R. B. et al. (1986) Mol. Gen. Genet. 203:237-244. Promoters that express in the embryo, pericarp, and endosperm are disclosed in U.S. Pat. No. 6,225,529 and PCT publication WO 00/12733. The disclosures for each of these are incorporated herein by reference in their entireties.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a miRNA expression construct within a particular plant tissue. Tissue-preferred promoters are known in the art. See, for example, Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590. In addition, the promoters of cab and rubisco can also be used. See, for example, Simpson et al. (1958) EMBO J. 4:2723-2729 and Timko et al. (1988) Nature 318:57-58.

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and rolD root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptll (neomycin phosphotransferase II) showed similar characteristics. Additional root-preferred promoters include the VfENOD-GRP3 gene promoter (Kuster et al. (1995) Plant Mol. Biol. 29(4):759-772); and rolB promoter (Capana et al. (1994) Plant Mol. Biol. 25(4):681-691). See also U.S. Pat. Nos. 5,837,876; 5,750,386; 5,633,363; 5,459,252; 5,401,836; 5,110,732; and 5,023,179. Phaseolin terminators can also be used (Murai et al. (1983) Science 23:476-482 and Sengopta-Gopalen et al. (1988) Proc. Natl. Aca. Sci USA 82:3320-3324.

The expression cassette containing the miRNA expression construct can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D) and sulfonylureas. Additional selectable markers include phenotypic markers such as beta-galactosidase and fluorescent proteins such as green fluorescent protein (GFP) (Su et al. (2004) Biotechnol. Bioeng. 85:610-9 and Fetter et al. (2004) Plant Cell 16:215-28), cyan fluorescent protein (CYP) (Bolte et al. (2004) J. Cell Sci. 117:943-54 and Kato et al. (2002) Plant Physiol. 129:913-42), and yellow fluorescent protein (PhiYFP™ from Evrogen; see, Bolte et al. (2004) J. Cell Sci. 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Nail. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the compositions presented herein.

Compositions comprising a cell, a transgenic plant cell, a transgenic plant, a transgenic seed, and a transgenic explant comprising a miRNA expression construct are further provided. In one embodiment, a cell, plant, plant cell or plant seed comprise a miRNA expression construct, wherein the most abundant form of miRNA produced from the miRNA expression construct is a 22-nt. It is recognized that the miRNA encoded by the miRNA expression construct can target any protein and/or gene family.

In further embodiments, cells, plant cells, plants or seeds comprise a miRNA expression construct comprising a miRNA precursor backbone further comprising a heterologous miRNA sequence and a heterologous star sequence. The miRNA precursor backbone can be from any plant. In some embodiments, the miRNA precursor backbone can be from a monocot (i.e. maize) or a dicot (i.e. soybean).

Plant cells that have been transformed to have a miRNA expression construct provided herein can be grown into whole plants. The regeneration, development, and cultivation of plants from single plant protoplast transformants or from various transformed explants is well known in the art. See, for example, McCormick et al. (1986) Plant Cell Rep. 5:81-84; Weissbach and Weissbach, In: Methods for Plant Molecular Biology, (Eds.), Academic Press, Inc. San Diego, Calif., (1988). This regeneration and growth process typically includes the steps of selection of transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. Two or more generations may be grown to ensure that expression of the desired phenotypic characteristic is stably maintained and inherited and then seeds harvested to ensure expression of the desired phenotypic characteristic has been achieved. In this manner, the compositions presented herein provide transformed seed (also referred to as “transgenic seed”) having a polynucleotide provided herein, for example, an miRNA expression construct, stably incorporated into their genome.

The miRNA expression constructs provided herein may be used for transformation of any plant species, including, but not limited to, monocots (e.g., maize, sugarcane, wheat, rice, barley, sorghum, or rye) and dicots (e.g., soybean, Brassica, sunflower, cotton, or alfalfa). Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffee spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed herein include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants provided herein are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments soybean plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Depending on the miRNA target sequence, the transgenic plants, plant cells, or seeds expressing an miRNA expression construct provided herein may have a change in phenotype, including, but not limited to, an altered pathogen or insect defense mechanism, an increased resistance to one or more herbicides, an increased ability to withstand stressful environmental conditions, a modified ability to produce starch, a modified level of starch production, a modified oil content and/or composition, a modified carbohydrate content and/or composition, a modified fatty acid content and/or composition, a modified ability to utilize, partition and/or store nitrogen, and the like.

Transformation protocols as well as protocols for introducing polynucleotide sequences into plants may vary depending on the type of plant or plant cell, i.e., monocot or dicot, targeted for transformation. Suitable methods of introducing polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechniques 6:923-926); and Lec1 transformation (WO 00/28058). Also see Weissinger et al. (1988) Ann. Rev. Genet. 22:421-477; Sanford et al. (1987) Particulate Sci. Technol. 5:27-37 (onion); Christou et al. (1988) Plant Physiol. 87:671-674 (soybean); McCabe et al. (1988) Biotechniques 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechniques 8:736-740 (rice); Klein et al. (1988) Proc. Natl. Acad. Sci. USA 85:4305-4309 (maize); Klein et al. (1988) Biotechniques 6:559-563 (maize); Klein et al. (1988) Plant Physiol. 91:440-444 (maize); Fromm et al. (1990) Biotechniques 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Rep. 9:415-418 and Kaeppler et al. (1992) Theor. Appl. Genet. 84:560-566 (whisker-mediated transformation); D′Halluin et al. (1992) Plant Cell 4:1495-1505 (electroporation); Li et al. (1993) Plant Cell Rep. 12:250-255 and Christou and Ford (1995) Ann. Bot. 75:407-413 (rice); Osjoda et al. (1996) Nat. Biotechnol. 14:745-750 (maize via Agrobacterium tumefaciens); all of which are herein incorporated by reference.

In specific embodiments, the miRNA expression construct disclosed herein can be provided to a plant using a variety of transient transformation methods. Such transient transformation methods include, but are not limited to, the introduction of the miRNA expression constructs or variants thereof directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. USA 91: 2176-2180 and Hush et al. (1994) J. Cell Sc. 107:775-784, all of which are herein incorporated by reference. Alternatively, the polynucleotides can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, miRNA expression constructs disclosed herein may be introduced into plants by contacting plants with a virus or viral nucleic acids. Generally, such methods involve incorporating a nucleotide construct of the disclosure within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Mol. Biotechnol. 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the miRNA expression constructs provided herein can be contained in a transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The miRNA expression construct is thereby integrated at a specific chromosomal position in the plant genome.

The cells that have been transformed may be grown into plants in accordance with conventional ways. See, for example, McCormick et al. (1986) Plant Cell Rep. 5:81-84.These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, transformed seed (also referred to as “transgenic seed”) having an miRNA expression construct disclosed herein, stably incorporated into their genome is provided.

When an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

### GENERAL METHODS

The following examples are provided to demonstrate different embodiments. It should be appreciated by those of skill in the art that the techniques disclosed in the examples which follow represent techniques discovered by the inventor to function well in the practice of the methods disclosed herein, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the presently disclosed methods.

The following abbreviations in the specification correspond to units of measure, techniques, properties, or compounds as follows: “sec” or “s” means second(s), “min” means minute(s), “h” or “hr” means hour(s), “μL” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “μg” means microgram(s), “rpm” means revolution(s) per minute, “EDTA” means ethylenediaminetetraacetic acid, “μE” means micro einstein(s), “PSI” means pounds per square inch, “SSC” means saline-sodium citrate, “SDS” means sodium dodecyl sulfate, “TBE” means Tris/Borate/EDTA, “QC” means quality control.

### EXAMPLE 1

### Constructs Producing siRNAs

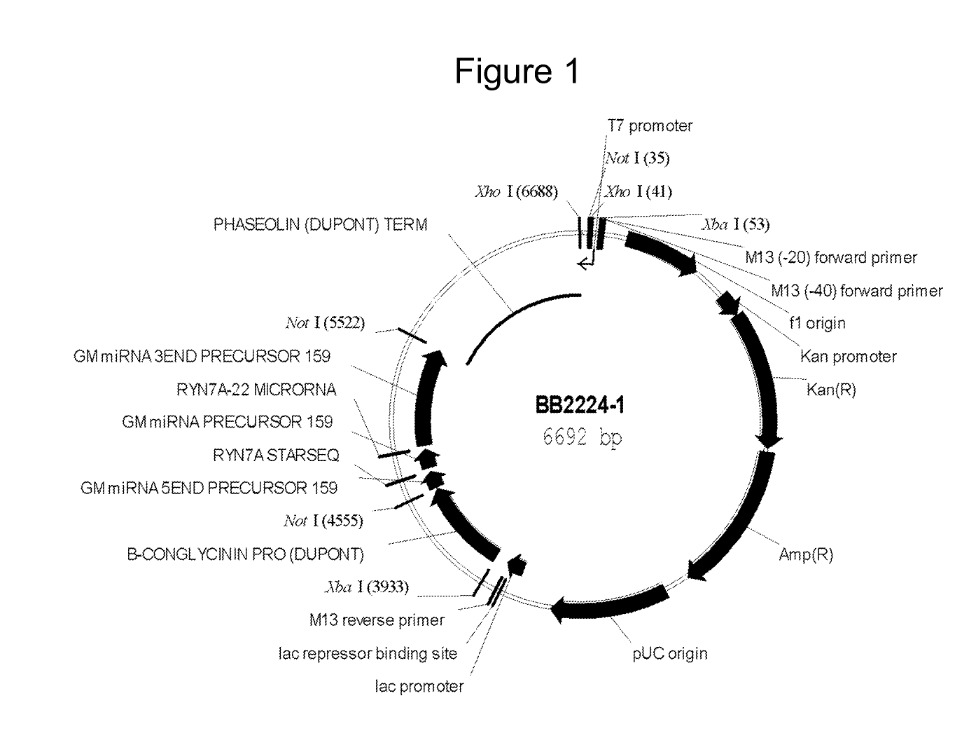

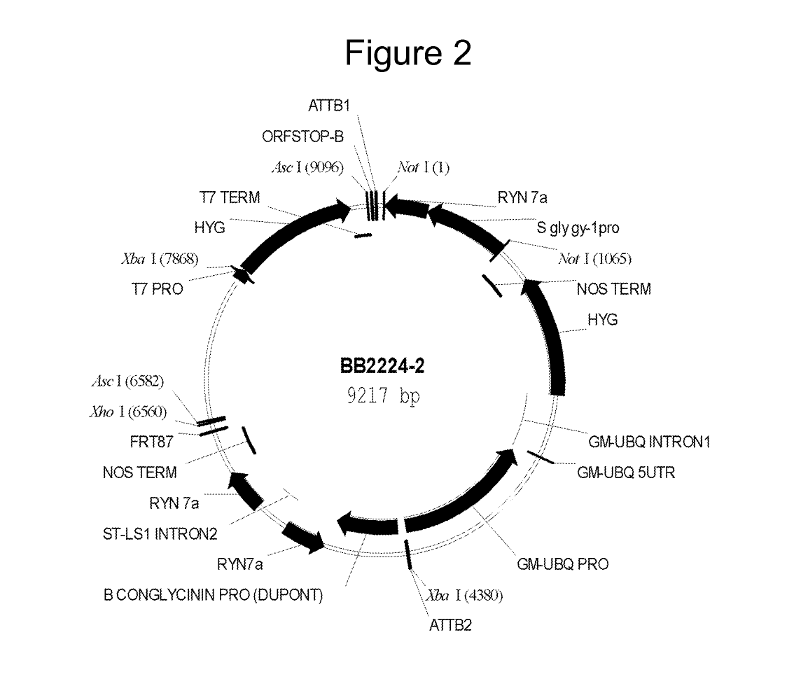

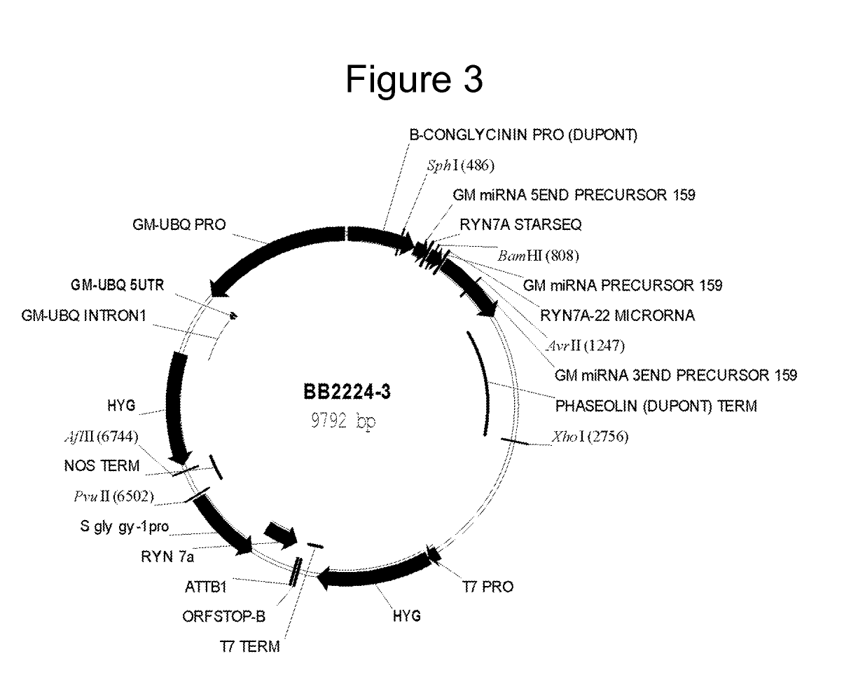

The Xbal/Xhol fragment of BB2224-1 (SEQ ID NO:1; FIG. 1) containing a beta-conglycinin promoter flanking the Glycine max 159 primary microRNA modified to produce a 22 base pair microRNA (SEQ ID NO:4) targeting the ryanodine 7 fragment (RYN7a; SEQ ID NO:5; US patent application 20120276554) followed by a phaseolin terminator was cloned into the Xbal/Xhol fragment of BB2224-2 (SEQ ID NO:2; FIG. 2). The BB2224-2 cassette includes a hygromycin phosphotransferase gene driven by a Glycine max ubiquitin promoter and followed by a NOS terminator that functions as a selectable marker in Glycine max transformation; the RYN7a fragment (SEQ ID NO:5) driven by a soybean glycinin 1 promoter; and a hygromycin phosphotransferase gene driven by a T7 promoter and followed by a T7 terminator for selection in bacteria. The resulting plasmid created was named BB2224-3 (SEQ ID NO:3, FIG. 3). RYN7a (SEQ ID NO:5) is a nucleotide fragment of the southern green stinkbug (Nezara viridula (Linnaeus)) sequence known as nezvi_{—}22408.WL.1 (SEQ ID NO:6) that has been shown to be active in a southern green stinkbug in vitro diet assay (co-owned, co-filed US Patent Application entitled “Compositions and Methods for Insecticidal Control of Stinkbugs”, attorney docket number PH15398).

Similar type of constructs as described above can be constructed containing maize specific elements (promoters and terminators active in maize) and expressing the RYN7a.

### EXAMPLE 2

### Transformation of Maize

Immature maize embryos from greenhouse donor plants are bombarded with a plasmid containing the silencing element of the disclosure operably linked to either a tissue specific, tissue selective, or constitutive promoter and the selectable marker gene PAT (Wohlleben et al. (1988) Gene 70:25-37), which confers resistance to the herbicide Bialaphos. In one embodiment, the constructs will express a long double stranded RNA of a target sequence or a fragment thereof. Such a construct can be linked to a promoter active in maize. Alternatively, the selectable marker gene is provided on a separate plasmid. Transformation is performed as follows. Media recipes follow below.

### Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising the silencing element of interest operably linked to either the tissue specific, tissue selective, or constitutive promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 mL 100% ethanol, and centrifuged for 30 seconds. Again the liquid is removed, and 105 μL 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in a particle gun. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/L Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5 inch pot) containing potting soil and grown for 1 week in a growth chamber, subsequently grown an additional 1-2 weeks in the greenhouse, then transferred to classic 600 pots (1.6 gallon) and grown to maturity.

Plants are monitored and scored for the appropriate marker, such as the control of a Pentatomidae plant pest, such as a N. viridula plant pest. For example, R₀ maize plants are fed to N. viridula second instar nymphs. Contamination and larval quality are monitored. Larval mass and survivorship are recorded for analysis. A one-way ANOVA analysis and a Dunnett's test is performed on the larval mass data to look for statistical significance compared to an untransformed negative control maize plant diet. N. viridula second instar nymph stunting is measured after feeding on two events and compared to growth of larvae fed on negative control plants.

In other assays, transgenic corn plants (R₀) generated are planted into 10-inch pots containing Metromix soil after reaching an appropriate size. After allowing the N. viridula second instar nymphs to feed on the plant, plants are removed from the soil and washed so that the relevant plant parts can be evaluated for larval feeding. Plant damage is rated using routine methods to score the level of damage.

Bombardment medium (560Y) comprises 4.0 g/L N6 basal salts (SIGMA C-1416), 1.0 mL/L Eriksson's Vitamin Mix (1000X SIGMA-1511), 0.5 mg/L thiamine HCl, 120.0 g/L sucrose, 1.0 mg/L 2,4-D, and 2.88 g/L L-proline (brought to volume with D-I H₂O following adjustment to pH 5.8 with KOH); 2.0 g/L Gelrite (added after bringing to volume with D-I H₂O); and 8.5 mg/L silver nitrate (added after sterilizing the medium and cooling to room temperature). Selection medium (560R) comprises 4.0 g/L N6 basal salts (SIGMA C-1416), 1.0 mL/L Eriksson's Vitamin Mix (1000X SIGMA-1511), 0.5 mg/L thiamine HCl, 30.0 g/L sucrose, and 2.0 mg/L 2,4-D (brought to volume with D-I H₂O following adjustment to pH 5.8 with KOH); 3.0 g/L Gelrite (added after bringing to volume with D-I H₂O); and 0.85 mg/L silver nitrate and 3.0 mg/L bialaphos (both added after sterilizing the medium and cooling to room temperature).

Plant regeneration medium (288J) comprises 4.3 g/L MS salts (GIBCO 11117-074), 5.0 mL/L MS vitamins stock solution (0.100 g nicotinic acid, 0.02 g/L thiamine HCl, 0.10 g/L pyridoxine HCl, and 0.40 g/L glycine brought to volume with polished D-I H₂O) (Murashige and Skoog (1962) Physiol. Plant. 15:473), 100 mg/L myo-inositol, 0.5 mg/L zeatin, 60 g/L sucrose, and 1.0 mL/L of 0.1 mM abscisic acid (brought to volume with polished D-I H₂O after adjusting to pH 5.6); 3.0 g/L Gelrite (added after bringing to volume with D-I H₂O); and 1.0 mg/L indoleacetic acid and 3.0 mg/L bialaphos (added after sterilizing the medium and cooling to 60° C.). Hormone-free medium (272V) comprises 4.3 g/L MS salts (GIBCO 11117-074), 5.0 mL/L MS vitamins stock solution (0.100 g/L nicotinic acid, 0.02 g/L thiamine HCl, 0.10 g/L pyridoxine HCl, and 0.40 g/L glycine brought to volume with polished D-I H₂O), 0.1 g/L myo-inositol, and 40.0 g/L sucrose (brought to volume with polished D-I H₂O after adjusting pH to 5.6); and 6 g/L bacto-agar (added after bringing to volume with polished D-I H₂O), sterilized and cooled to 60° C.

### EXAMPLE 3

### Aqrobacterium-Mediated Transformation of Maize

For Agrobacterium-mediated transformation of maize with a silencing element disclosed herein, the method of Zhao is employed (U.S. Pat. No. 5,981,840, and PCT patent publication WO98/32326; the contents of which are hereby incorporated by reference). Such as a construct can, for example, express a long double stranded RNA of a target sequence. Such a construct can be linked to the dMMB promoter. Briefly, immature embryos are isolated from maize, and the embryos contacted with a suspension of Agrobacterium, where the bacteria are capable of transferring the polynucleotide comprising the silencing element to at least one cell of at least one of the immature embryos (step 1: the infection step). In this step, the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period, an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants. Assays for insecticidal activity can be performed as described above in Example 3.

### EXAMPLE 4

### Transformation and Regeneration of Soybean (Glycine max)

Transgenic soybean lines are generated by the method of particle gun bombardment (Klein et al., Nature (London) 327:70-73 (1987); U.S. Pat. No. 4,945,050) using a BIORAD Biolistic PDS1000/He instrument and either plasmid or fragment DNA.

Integration of DNA into the soybean genome after particle gun-mediated transformation may be random, or it may be through site-specific integration (SSI), achieved by recombinase-mediated cassette exchange (RMCE) at a previously created transgenic target site (U.S. Pat. No. 7,102,055 issued Sep. 5, 2006). Recombinase Medicated DNA Casette exchange RMCE using different recombinase systems have been achieved successfully in several plants (Nanto K et al. (2005) Agrobacterium-mediated RMCE approach for gene replacement, Plant Biotechnol J 3:203-214; Louwerse JD et al. (2007) Stable recombinase-mediated cassette exchange in Arabidopsis using Agrobacterium tumefaciens, Plant Physiol 145:1282-1293; Li Z et al. (2009) Site-specific integration of transgenes in soybean via recombinase-mediated DNA cassette exchange, Plant Physiol 151:1087-1095). Groups of transgenes can be stacked to the same site through multiple rounds of RMCE (Li Z et al. (2010) Stacking multiple transgenes at a selected genomic site via repeated recombinase-mediated DNA cassette exchanges, Plant Physiol 154:622-631). Taking advantage of reversible DNA cassette exchange in RMCE, an RMCE product can be used as a new target for subsequent SSI transformation.

The transgenic target site for RMCE may contain a promoter followed by recombination sites surrounding a selectable marker gene such as the hygromycin phosphotransferase (HPT) gene, with or without additional components. After bombardment with donor DNA, the target DNA previously integrated into the soybean genome recombines with the donor DNA at recombination sites such as FRT1 and FRT87 with the help of a transiently expressed recombinase such as the FLP recombinase. The portion of the DNA cassette in the target which contains the original selectable marker gene flanked by dissimilar recombination sites such as FRT1 and FRT87 is replaced by the donor DNA cassette flanked by the same FRT1 and FRT87 sites, resulting in site-specific specific integration of the donor cassette to the exact same genomic site of the target. The promoter existing upstream of the recombination sites in the transgenic target remains after RMCE to regulate expression of the new selectable marker gene delivered to the site as part of the donor cassette. Successful RMCE events may be identified by chemical selection for cells expressing the selectable marker gene of the donor.

Culture media and stock solutions

The following stock solutions and media are used for transformation and regeneration of soybean plants:

Stock solutions:
Sulfate 100× Stock: 37.0 g MgSO₄.7H₂O, 1.69 g MnSO₄.H₂O, 0.86 g ZnSO₄.7H₂O, 0.0025 g CuSO₄.5H₂O
Halides 100× Stock: 30.0 g CaCl₂.2H₂O, 0.083 g KI, 0.0025 g CoCl₂.6H₂O
P, B, Mo 100× Stock: 18.5 g KH₂PO₄, 0.62 g H₃BO₃, 0.025 g Na₂MoO₄.2H₂O
Fe EDTA 100× Stock: 3.724 g Na₂EDTA, 2.784 g FeSO₄.7H₂O
2,4-D Stock: 10 mg/mL Vitamin
B5 vitamins, 1000× Stock: 100.0 g myo-inositol, 1.0 g nicotinic acid, 1.0 g pyridoxine HCl, 10 g thiamine HCL

### Media (per Liter):

SB199 Solid Medium: 1 package MS salts (Gibco/BRL—Cat. No. 11117-066), 1 mL B5 vitamins 1000× stock, 30 g Sucrose, 4 ml 2,4-D (40 mg/L final concentration), pH 7.0, 2 gm Gelrite
SB1 Solid Medium: 1 package MS salts (Gibco/BRL—Cat. No. 11117-066), 1 mL B5 vitamins 1000× stock, 31.5 g Glucose, 2 mL 2,4-D (20 mg/L final concentration), pH 5.7, 8 g TC agar
SB196: 10 mL of each of the above stock solutions 1-4, 1 mL B5 Vitamin stock, 0.463 g (NH₄)₂SO₄, 2.83 g KNO₃, 1 mL 2,4 D stock, 1 g asparagine, 10 g Sucrose, pH 5.7
SB71-4: Gamborg's B5 salts, 20 g sucrose, 5 g TC agar, pH 5.7
SB103: 1 pk. Murashige & Skoog salts mixture, 1 mL B5 Vitamin stock, 750 mg MgCl₂ hexahydrate, 60 g maltose, 2 g gelrite, pH 5.7
SB166: SB103 supplemented with 5 g per liter activated charcoal.

### Soybean Embryoqenic Suspension Culture Initiation

Pods with immature seeds from available soybean plants 45-55 days after planting are picked, removed from their shells and placed into a sterilized magenta box. The soybean seeds are sterilized by shaking them for 15 min in a 5% Clorox solution with soap or other surfactants at 1 drop per 100 mL solution. Seeds are rinsed with sterile distilled water, and those less than 4 mm are placed on a sterile surface under microscope. The small ends of seeds are cut, and the cotyledons are pressed out of the seed coats. Cotyledons are transferred to plates containing SB199 medium (25-30 cotyledons per plate) for 2 weeks, then transferred to SB1 for 2-4 weeks. Plates are wrapped with fiber tape and cultured for 8 weeks in growth chamber room with temperature set at 24.4-26° C. and light on a 16:8 h day/night photoperiod at an intensity of 45-65 μE/m²/s . After this time, secondary embryos are cut and placed into SB196 liquid medium for 7 days.

### Culture Conditions

Soybean embryogenic suspension cultures are maintained in 50 mL liquid medium SB196 on a rotary shaker at a speed of 100-150 rpm. The cultures are set in a growth chamber with temperature set at 24.4-26° C. and light on a 16:8 h day/night photoperiod at intensity of 80-100 μE/m²/s for liquid culture and 80-120 μE/m²/s for maturation and germination. Cultures are subcultured every 7-14 days by inoculating up to ½ dime size quantity of tissue into 50 mL of fresh liquid SB196.

### Preparation of DNA for Bombardment

In particle gun bombardment procedures it is possible to use purified (1) entire plasmid DNA or (2) DNA fragments containing only the recombinant DNA expression cassette(s) of interest. For every bombardment experiment, 85 μL of suspension is prepared containing 1 to 90 picograms (pg) of plasmid DNA per base pair of DNA. To prepare for an SSI transformation, the donor plasmid is mixed with plasmid DNA containing the FLP recombinase gene cassette in a ratio such as 3:1. Both recombinant DNA plasmids are co-precipitated onto gold particles as follows. The DNAs in suspension are added to 50 μL of a 10-60 mg/mL 0.6 μm gold particle suspension and then combined with 50 μL CaCl₂ (2.5 M) and 20 μL spermidine (0.1 M). The mixture is vortexed for 5 sec, spun in a microcentrifuge for 5 sec, and the supernatant removed. The DNA-coated particles are then washed once with 150 μL of 100% ethanol, vortexed and spun in a microcentrifuge again, then resuspended in 85 μL of anhydrous ethanol. Five μL of the DNA-coated gold particles are then loaded onto each macrocarrier disk.

Tissue Preparation and Bombardment with DNA

Approximately 100-200 mg of two-week-old suspension culture is placed in an empty 60 mm×15 mm petri plate and the residual liquid removed from the tissue using a pipette. The tissue is placed about 3.5 inches away from the retaining screen. Membrane rupture pressure is set at 650 psi and the bombardment chamber of the particle gun is evacuated to −28 inches of Hg prior to bombardment. Typically, each plate of tissue is bombarded once.

### Selection of Transformed Embryos and Plant Regeneration

After bombardment, tissue from each bombarded plate is divided and placed into one to two flasks of SB196 liquid culture maintenance medium per plate of tissue, one flask per 100 mg tissue. Seven days post bombardment, the liquid medium in each flask is replaced with fresh SB196 culture maintenance medium supplemented with 100 ng/ml selective agent (selection medium). For selection of transformed soybean cells after random transformation or RMCE, the selective agent used can be a sulfonylurea (SU) compound with the chemical name, 2-chloro-N-((4-methoxy-6-methy-1,3,5-triazine-2-yl)aminocarbonyl) benzenesulfonamide (common names: DPX-W4189 and chlorsulfuron). Chlorsulfuron is the active ingredient in the DuPont sulfonylurea herbicide, GLEAN®. The selection medium containing SU is replaced every two weeks for 8 weeks. After the 8 week selection period, islands of green, transformed tissue are observed growing from untransformed, necrotic embryogenic clusters. The putative transgenic randomly integrated or RMCE events are isolated and kept in SB196 liquid medium with SU at 100 ng/ml for another 5 weeks with media changes every 1-2 weeks to generate new, clonally propagated, transformed embryogenic suspension cultures. Embryos spend a total of around 13 weeks in contact with SU. Suspension cultures are subcultured and maintained as clusters of immature transgenic embryos and also regenerated into whole plants by maturation and germination of individual somatic embryos.

Transgenic somatic embryos become suitable for germination after four weeks on maturation medium (1 week on SB166 followed by 3 weeks on SB103). They are then removed from the maturation medium and dried in empty petri dishes, or with a small amount of medium, for approximately seven days. The dried embryos are then planted in SB71-4 medium where they are allowed to germinate under the same light and temperature conditions as described above. Germinated embryos are allowed to develop into small plantlets and are then transferred to potting medium and grown to maturity for seed production.

### EXAMPLE 5

### Transformation of Plant Cells with Constructs Producing siRNAs Targeting Insect Genes

Soybean cells were transformed with plasmid BB2224-3 as described in Example 4, and transgenic plants were generated. Transgenic soybean events can be evaluated for insecticidal activity as described in Example 7.

Maize cells can be transformed with plasmids or DNA fragments expressing the RYN7a as described in Example 1. Transgenic maize events can then be evaluated for insecticidal activity.

### EXAMPLE 6

### Detection of siRNAs

Methods used to detect siRNAs in transformed plant tissue include, but are not limited to: Northern Blot Analysis of small RNAs and Illumina Sequencing of small RNAs as described below.

### Northern Blot Analysis of Small RNAs

Total RNA can be isolated from transformed plant tissue using RNAzol (Molecular Research Center; catalog # RN190) according to the manufacturer's instructions and run on a 15% TBE-Urea polyacrylamide gel (Biorad). RNA can be transferred to a Positive Charged Nylon Membrane (Roche) using a Mini Trans-Blot cell (Bio-Rad) according to the manufacturer's instructions. The RNA can then be crosslinked to the membrane in a Stratalinker (Stratagene) using one cycle on Auto-Energy.

Negative strand RNA probes can be made using a DIG RNA labeling Kit (Roche; catalog #11175025910) according to the manufacturer's protocol. The membrane will can be prehybridized for at least one hour in DIG Easy Hyb buffer (Roche Cat # 116035580) at 37-45° C. After an hour, the prehybridization solution is removed and DIG Easy Hyb buffer (Roche Cat #116035580) including the probe is added and the membrane is allowed to hybridize overnight at 37-45° C. After hybridization, the blot can be washed using the DIG Wash and Block Buffer Set (Roche Cat #11585762001) at two stringencies (2×SSC, 0.1%SDS and 2×SSC, 0.1%SDS) according to the manufacturer's instructions, and signal can be detected using CDP-Star (Roche Cat #11685672001).

### Illumina Sequencing of Small RNAs.

Small RNA sequences can be generated according to smRNA-seq methods provided by Illumina Inc. (San Diego, CA) for the HiSeq 2000 sequencing instrument. In brief, 1 μg of total RNA per sample was used to generate smRNA-seq libraries using the TruSeq smRNA-seq kit (Illumina). RNA 3′ and 5′ adapters are ligated in consecutive reactions with T4 RNA ligase. Ligated RNA fragments can be primed with an adapter specific RT primer and reverse transcribed with Superscipt II reverse transcriptase (Life Technologies). Fragments can be bar-coded and amplified 11 cycles with adapter specific primers. Resulting cDNA libraries can be separated on a 6% TBE gel and library fragments with inserts of 18-25 bp can be excised. Gel slices can be shredded and libraries can be recovered by elution. Libraries can be validated by QC on a Agilent Bioanalyzer HiSens DNA chip (Agilent Technologies Inc. Santa Clara, Calif.) and can be pooled in equal molar ratios for sequencing. Forty-eight sample pools can be sequenced on one lane of a HiSeq 2000 for 50 cycles according to Illumina protocols. Resulting 50 bp sequence reads can be trimmed for read-though adapter sequence.

### EXAMPLE 7

### Bioassay of Soybean Plants

After transformation, transgenic soybean plants can be grown in the greenhouse and seeds can be harvested from these transformed plants and designated as T1 seeds. T1 seeds can be chipped manually, and DNA extracted from the chips can be used to determine zygosity using a quantitative PCR assay. Homozygous seeds can be sown in 2.5 inch pots, maintained in the growth chambers in 16:8 (light:dark) cycle in an insecticide free environment. After about 4 weeks, these plants can be transplanted to a larger pot and maintained at 14:10 (light:dark) cycle for 2 weeks. After two weeks, the plants can be maintained in 12:12 (light:dark) cycle to induce flowering and delivered for bioassay at R3 stage. Fertilizer can be provided as needed, and chambers are maintained at 50% relative humidity. Ten second instar southern green stinkbugs can be used to infest soybean pods at various stages: R3 (beginning pod), R4 (full pod), R5 (beginning seed), R6 (full seed) and R7 (beginning maturity). Insects can be maintained on the pods using enclosures. Developmental stage, stunting (% control as outlined in example 5) and mortality can be recorded at 8-10 days after initial infest of the transgenic soybean pods.

What is claimed is:

1. A method for reducing expression of at least one target sequence, said method comprising:
(a) expressing in a plant a recombinant DNA construct comprising:
(i) a first polynucleotide sequence comprising a plant-specific promoter operably linked to a nucleotide sequence encoding a pre-miRNA, wherein said pre-miRNA comprises a 22 nucleotide mature miRNA; and
(ii) a second polynucleotide sequence comprising at least one target sequence that can be cleaved by the mature mi-RNA processed by the pre-miRNA of (i),
wherein said plant processes said pre-miRNA into mature miRNA and;
(b) eliciting production of of secondary siRNAs in planta by the mature miRNA;
wherein exposing a target organism to said plant comprising the secondary siRNAs of step (b), reduces expression of at least one target sequence in said target organism.

2. The method of claim 1, wherein the second polynucleotide sequence is operably linked to a nucleotide comprising a second plant-specific promoter.

3. The method of claim 1, wherein said target sequence is derived from an insect.

4. The method of claim 3, wherein said insect is from an order selected from the group consisting of Pentatomidae and Chrysomelidae.

5. The method of claim 4, wherein the insect from order Pentatomidae is Nezara viridula.

6. The method of claim 4, whererin the insect from order Chrysomelidae is Diabrotica virgifera virgifera.

7. The method of claim 1, wherein said first polynucleotide segment comprises the nucleotide sequence set forth in SEQ ID NO:4.

8. The method of claim 1, wherein said mature miRNA has sufficient sequence complementary to said at least one target sequence whose level of RNA is to be reduced but does not have sufficient sequence complementary to any RNAs of a plant expressing the recombinant DNA construct.

9. The method of claim 1, wherein the target organism is exposed to the secondary siRNA by ingestion of said plant or a part of said plant.

10. A recombinant DNA construct comprising:
(a) a first polynucleotide sequence comprising a plant-specific promoter operably linked to a nucleotide sequence encoding a pre-miRNA, wherein said pre-miRNA comprises a 22 nucleotide mature miRNA; and
(b) a second polynucleotide sequence comprising at least one exogenous target sequence that can be cleaved by the mature mi-RNA processed by the pre-miRNA of (a);
wherein the mature miRNA elicits the production of secondary siRNAs.

11. A plant cell comprising the recombinant DNA construct of claim 10.

12. A plant or a plant part thereof comprising:
(a) a first recombinant DNA construct comprising a first plant-specific promoter operably linked to a polynucleotide encoding a first portion of a pre-miRNA, said first portion of a pre-miRNA comprising a first polynucleotide segment of 22 nucleotides; and
(b) a second recombinant DNA construct comprising a second plant-specific promoter operably linked to a polynucleotide encoding a second portion of a pre-miRNA, said second portion of a pre-miRNA comprising a second polynucleotide segment complementary to said first polynucleotide segment;
wherein said first polynucleotide segment has sufficient sequence complementary to at least one target sequence whose level of RNA is to be reduced but does not have sufficient sequence complementary to any RNAs of a plant expressing the recombinant DNA constructs; and further wherein said first polynucleotide sequence, when processed into a mature miRNA, elicits the production of secondary siRNAs.

13. A method for reducing expression of at least one target sequence, said method comprising:
(a) providing the plant or plant part thereof of claim 12;
(b) expressing the recombinant DNA constructs in the plant or plant part thereof, wherein said plant or plant part thereof processes said pre-miRNA into mature miRNA;
(c) wherein said mature miRNA elicites the production of secondary siRNAs in planta; and
(d) exposing a target organism to said plant or plant part thereof comprising secondary siRNAs of step (c), whereby exposure to the secondary siRNAs reduces expression of at least one target sequence.

14. A plant or plant part thereof comprising:
(a) a first polynucleotide comprising a plant-specific promoter operably linked to a nucleotide sequence encoding a pre-miRNA, wherein said pre-miRNA comprises a 22 nucleotide mature miRNA; and
(b) a second polynucleotide sequence comprising at least one exogenous target sequence that can be cleaved by the mature mi-RNA processed from the pre-miRNA of (a);
wherein said mature miRNA, elicits the production of secondary siRNAs.
